(19) United States
(12) Patent Application Publication
(10) Pub. No.: US 2004/0141967 A1
(43) Pub. Date: Jul. 22, 2004

(54) Methods and compositions for treating macrophage-mediated diseases

(76) Inventor: Jan G.J. van de Winkel, PR Odijk (NL)

Correspondence Address: LAHIVE & COCKFIELD, LLP., 28 STATE STREET, BOSTON, MA, 02109, US

(21) Appl. No.: 10/627,307

(22) Filed: Jul. 25, 2003

Related U.S. Application Data
(63) Continuation of application No. 09/251,570, filed on Feb. 17, 1999, now abandoned.
(60) Provisional application No. 60/074,967, filed on Feb. 17, 1998.

(51) Int. Cl. A61K 48/00; A61K 39/395

(52) U.S. Cl. 424/144.1; 424/93.2

(57) ABSTRACT

The invention provides methods and compositions for selectively targeting macrophages in a localized area. The compositions of the invention include an Fc receptor binding agent, and a toxic or a detectable agent. Methods for depleting or inhibiting the activity of macrophages using the compositions of the invention are disclosed. The compositions of the invention can be used therapeutically and diagnostically.

### RELATED APPLICATIONS

This application is a continuation of U.S. Ser. No. 09/251,570, filed Feb. 17, 1999, which claims priority to U.S. Serial No. 60/074,967, filed on Feb. 17, 1998, the contents of which are incorporated herein by reference.

### BACKGROUND OF THE INVENTION

In normal human skin, two compartmental layers can be distinguished. The upper layer, the epidermis, consists of keratinocytes, Langerhans' cells and T cells. The lower layer, the dermis, consists of fibroblasts, endothelial cells, dendritic cells, T cells, mast cells and macrophages.

The skin serves as an important boundary between the internal milieu and the environment. It primarily prevents contact with potentially harmful antigens. In case of antigen/pathogen penetration, an inflammatory response is induced in vivo to eliminate the antigen. This response leads to a dermal infiltrate, the composition of which depends on the type of response induced, but consists predominantly of T cells, polymorphonuclear cells, and monocytes (Williams, I. R., and Kupper, T. S. (1996.) Life Sci. 58: 1485-1507; Stingl, G. (1993) Recent Results Cancer Res. 128: 45-57). In addition, allergen nonspecific stimuli like tissue injury and ultraviolet light can also trigger an inflammatory response. In general, mechanisms underlying the allergen non-specific response are also employed during the effector phase of the allergen-specific response.

Macrophages are bone-marrow derived cells with great heterogeneity and versatility. These cells can produce a wide range of mediators and exert a multitude of biological functions (Ganz, T. (1993) New Horiz. 1: 23-27). Their phenotype and function is largely determined by local environment, whereas macrophage-derived mediators can thereupon influence their microenvironment. This microenvironment leads to regionally different subsets of macrophages and even locally, different macrophage subsets can be present (Gordon, S. (1995) Bioessays 17: 977-986). These cells are potent effector cells producing reactive oxygen products and proteolytic enzymes, which can directly damage tissue (Laskin, D. L., and Pendino, K. J. (1995) Annu Rev Pharmacol. Toxicol. 35:655-677). Under normal conditions, macrophages regulate proliferation of extracellular matrix-forming cells like fibroblasts in skin (Gonzalez-Ramos, A. et al. (1996) J. Invest. Dermatol. 106: 305-311). In addition, macrophages can exert important immunoregulatory functions and in this way play a crucial role in controlling and directing immune responses (Gordon, S. (1995) Bioessays 17:977-986; Thepen, T. et al. (1994) Ann. N.Y. Acad. Sci. 725:200-206). These cells can serve as antigen presenting cells, but also directly inhibit antigen presentation by dendritic cells (Holt, P. G. et al. (1993) J. Exp. Med. 177:397-407). Proliferation, phenotype and thus function of T cells, and thereby the type of immune response induced, can be influenced by macrophages.

Skin macrophages have been shown to play an important role in the regulation of cell growth of different non-hematopoietic cells (such as fibroblasts and keratinocytes), as well as in the functioning of T cells and dendritic cells. Under “steady state” conditions, the number of skin macrophages is relatively low. However, under various pathological conditions (for example, in active lesions), the number of macrophages is significantly increased. Tissue macrophages and infiltrating monocytes have been associated with modified fibroblast and keratinocyte function in inflammatory lesions, as well as aberrant functioning of T cells and/or dendritic cells.

Ultraviolet light exposure has been shown to induce a population of macrophages in the skin that, in contrast to Langerhans' cells, are capable of activating autoreactive T cells. Deregulated macrophage function has been directly correlated with abnormal cutaneous immune responsiveness in various diseases, including cutaneous T cell lymphoma (mycosis fungoides), psoriasis, atopic dermatitis, and cutaneous lupus erythematosus (Cooper, K. D. et al. (1993) J. Invest. Dennatol. 101: 155-163; Gonzalez-Ramos, A. et al. (1996) J. Invest. Dennatol. 106: 305-311). These cells can also activate resident and inflammatory macrophages, resulting in a “vicious circle” which maintains the cutaneous inflammation. In addition to the regulation of cell function, macrophages are potent producers of toxic compounds such as oxygen radicals and proteolytic enzymes. These toxic compounds have been shown to cause direct tissue damage.

### SUMMARY OF THE INVENTION

The present invention provides methods and compositions for selectively targeting cytotoxic compounds via Fc receptors to monocyte-derived phagocytic cells (i.e., macrophages). The invention can thus be used to selectively reduce the number or activity of a population of macrophages within a localized area, such as the skin, joints or lungs.

In one embodiment, the invention provides a macrophage-binding compound which contains at least a first portion which binds to an Fc receptor present on a macrophage, and at least a second portion which kills or inhibits the function of the macrophage. The portion which binds to the Fc receptor can include any molecule capable of Fc receptor binding, such as an antibody, a peptide (e.g., peptide mimetic) or a chemical compound. In one embodiment, the Fc receptor binding portion is an antibody or antibody fragment (e.g., an Fab, Fab′, F(ab′)₂, Fv, or a single chain Fv). In a preferred embodiment, the anti-Fc receptor antibody or antibody fragment is “humanized” (e.g., has at least a complementarity determining region (CDR) or a portion thereof derived from a non-human antibody (e.g., murine) with the remaining portion(s) are human in origin). In another preferred embodiment, the anti-Fc receptor antibody or antibody fragment is a human monoclonal antibody (e.g., an antibody produced in a mouse genetically-engineered to express a completely human antibody). Also included among these embodiments are compounds (e.g. peptides or chemical species) which “mimic” the binding of such anti-Fc receptor antibodies (Jenks et al. J. Natl. Cancer inst. (1992) 84(2):79; Saragovi et al. Science (1991) 253:792; Hinds et al. J. Med. Chem. (1991) 34:1777-1789; Fassina Immunomethods (1994) 5:121-129). In another embodiment, the Fc receptor binding portion of the macrophage-binding compound is a cyanin composition, such as the fluorescent dye Cy5.18.OSu (referred to herein as “Cy5”), which binds with high affinity and specificity to the FcγRI receptor present on macrophage cells. The cyanin compositions can include at least two moieties: a cyanin succinimidyl ester and a phycobilisome protein, e.g., PE.

The Fc receptor recognized by the macrophage-binding compounds of the invention can be an IgG receptor, e.g., an Fc-gamma receptor (FcγR), such as FcγRI (CD64), FcγRII(CD32), and FcγRIII (CD 16), or an IgA receptor, e.g., an FcαR (e.g., FcαRI, CD89). The Fc receptor is preferably located on the surface of a macrophage, e.g., a skin macrophage, so that it is capable of being recognized and bound by the compound. In a preferred embodiment, the anti-Fc receptor binding portion of the macrophage-binding compound binds to an Fc receptor at a site which is distinct from that bound by endogenous immunoglobulins (e.g., IgGs or IgAs). Therefore, the binding of the macrophage-binding compounds to the Fc receptor is not blocked by physiological levels of immunoglobulins.

A preferred Fc receptor on a macrophage for targeting is the high affinity Fcγ receptor, FcγRI. Thus, in one embodiment, the anti-Fc receptor binding portion of the macrophage-binding compounds of the invention comprise an anti-FcγRI antibody, or a fragment thereof. Exemplary anti-FcγRI antibodies include mAb 22, mAb 32, mAb 44, mAb 62 and mAb 197. In preferred embodiments, a humanized form of such anti-FcγRI receptor antibodies are used, such as humanized monoclonal antibody 22 (H22), or a fragment thereof.

The portion of the macrophage-binding compound which kills or modulates (e.g., reduces) the activity of a macrophage (the anti-macrophage agent) can be selected from suitable cytotoxins or drugs. For example, the anti-macrophage agent can be Gelonin, Saporin, Onconase, Exotoxin A, Ricin A, dichloromethylene diphosphonate (CL2MDP), or derivatives thereof. In one embodiment, the anti-macrophage agent is directly linked to the anti-Fc receptor binding portion of the macrophage-binding compound. In another embodiment of the invention, the anti-macrophage agent is indirectly linked to the anti-Fc receptor binding portion. For example, the anti-macrophage agent can be encapsulated within a liposome which is linked to the anti-Fc receptor binding portion.

Macrophage-binding compounds of the invention can be used in a variety of therapeutic and diagnostic methods. In one embodiment, these compounds are used to diagnose a disease characterized by abnormal numbers or function of macrophages. The method involves contacting or administering to a test area, or a cultured sample, the macrophage-binding compound under conditions that allow for binding of the compound to macrophages present in the sample. Binding of the compound can then be detected as an indication of the presence (e.g., number) and/or function of macrophages in the sample. For example, a statistically significant elevated level of Fc receptor protein specifically detected, indicating an increase in the number of macrophages, can be indicative of a disease. The test area or sample can be from, e.g., the skin (e.g., human skin) or other tissue containing macrophage cells.

In another embodiment, the macrophage-binding compounds are used to treat a disease involving proliferation and/or abnormal functioning of macrophages. Upon contacting macrophage-binding compounds with an area needing treatment, the compounds bind to macrophages via their Fc receptors and kill or reduce the activity of these cells. Accordingly, a broad variety of diseases involving macrophages (e.g., macrophage proliferation and/or abnormal functioning) can be treated, prevented or diagnosed using the compounds of the invention. Such diseases can be of intrinsic origin (e.g., autoimmune disease), or extrinsic origin (e.g., contact hypersensitivity, Polymorphic Light Eruption (PLE), and irritants reactions). Skin disease can furthermore be a manifestation of a more systemic disease like atopic dermatitis (AD) in the case of atopy, and systemic lupus erythematosus. A non-limiting list of the diseases that can be treated with the compositions and methods of the present invention include autoimmune diseases, respiratory diseases, infectious diseases, dermatological diseases and inflammatory conditions. Specific examples of such diseases include, but are not limited to, psoriasis, atopic dermatitis, multiple sclerosis, scleroderma, cutaneous lupus erythematosis, rheumatoid arthritis, Human Immunodeficiency Virus (HIV) infections, Chronic Polymorphic Light Dermatosis (CPLD), Chronic Obstructive Pulmonary Diseases (COPD), e.g., allergic asthma and Sarcoidosis, Wegener's Granulomatosis, and inflammatory conditions, such as skin lesions (e.g., open wounds or burn wounds). Additionally, the methods and compositions of the invention can be used in vitro to diagnose such diseases, or for research purposes (e.g., to study the pathological role of macrophages in such diseases). In other embodiments, the macrophage-binding compounds of the invention can be used in cosmetic applications, e.g., to delay or ameliorate the aging process.

When used in vivo for therapeutic purposes, macrophage binding compounds of the invention can be locally administered (e.g., topically, intradermally, subcutaneously or by inhalation as an aerosol) to a selected area in an amount effective to deplete, or reduce the activity of macrophages within the area of administration. In certain embodiments, the macrophage binding compound can include a photosensitizing agent which is inactive when administered (e.g., systemically, topically, intramuscularly), but is activated by exposure to light (e.g., visible or UV light). Similarly, the macrophage binding compounds can include an Fc binding agent linked to a therapeutic (or diagnostic reagent) via a photocleavable linkage, which upon light exposure releases the reagent. These compounds allow for controlled killing or inactivation of macrophages only within selected tissues exposed to light.

The present invention further provides compositions, e.g., a pharmaceutical compositions, containing macrophage-binding compounds along with an acceptable carrier or diluent, for use in the methods described above.

Other features and advantages of the invention will be apparent from the following figures, detailed description, examples and claims.

### BRIEF DESCRIPTION OF THE DRAWINGS

FIG. 1 is a bar graph depicting the percentage of [³H]-Thymidine incorporation of cultured U937 or IIA1.6 cells grown in the presence or absence of varying concentrations of a CD64-immunotoxin (H22-Ricin A, H22-R, or 197-Ricin A, 197-R) as compared with that of medium control (±SEM). U937 cells were cultured either with (black bars) or without (gray bars) IFNγ in the presence of the indicated concentrations of H22-R (panel A) or 197-R (panel B). In the lower panels C and D, IIA1.6 cells, either transfected with hFcγRI (black bars) or non-transfected (gray bars), were incubated with varying concentrations of H22-R (panel C) or 197-R (panel D).

FIG. 2 is a scan of propidium iodide fluorescence of U937 cells as these cells undergo apoptosis after incubation with varying concentrations of H22-R. Nuclear fragmentation was analyzed with propidium iodide staining and subdiploid nuclei are indicated by bars. Numbers above bars specify percentage of subdiploid, hence apoptotic nuclei. Con=Control.

FIGS. 3A and B are graphs showing the effect of a single intradermal injection of an immunotoxin on inflammatory cells in skin with respect to time. Data points represent mean number of cells per mm² (±SEM) and data points represent the average of >3 experiments. Depicted are the kinetics of hFcγRI-expressing cells (filled square, FIG. 3A), macrophages (blank square, FIG. 3A), T cells (filled square, FIG. 3B), and dendritic cells (blank square, FIG. 3B).

FIGS. 4A-4B are graphs showing a decrease in local skin temperature upon intradermal injection of an immunotoxin. FIG. 4A depicts local skin temperature readings (±SEM) of SLS treated hFcγRI transgenic mice after a single injection with IT (●) (n=6) or vehicle control (O) (n=6). FIG. 4B shows temperature course (±SEM) of SLS treated hFcγRI-transgenic mice, injected with either IT (O) (n=6), or vehicle control (●) (n=6). Local skin temperature was monitored daily, and upon increase, animals were re-injected at the same site (days marked with *).

### DETAILED DESCRIPTION OF THE INVENTION

Abnormal macrophage function, including aberrant proliferation and/or activity, has been implicated in a variety of disorders, such as dermatological diseases, autoimmune diseases, infectious diseases and inflammatory conditions. To date, methods of localized ablation of macrophages using cytotoxic agents, e.g., immunotoxins, have had limited efficacy. The present invention provides methods and compositions for diagnosing, treating and preventing such disorders by selectively depleting and/or inhibiting the activity of macrophages within a localized area. Cells are depleted (e.g., killed) and/or inhibited (e.g., activity reduced) by targeting a toxic agent to them via their Fc receptors. For example, studies described herein demonstrate the use of a macrophage-binding compound consisting of an anti-Fc receptor binding portion, e.g., a humanized antibody against a human FcγRI receptor, conjugated to a toxin, e.g., Ricin A, to selectively eliminate macrophages in vivo in transgenic mice expressing human FcγRI. As used herein, the terms “macrophage” and “monocyte-derived phagocytic cell” shall be used interchangeably.

Accordingly, in one embodiment, the invention provides a macrophage-binding compound comprising an agent which binds to an Fc receptor present on a macrophage and an agent which kills or inhibits the activity of the macrophage which is bound. Suitable components for binding Fc receptors include, for example, proteins (e.g., anti-FcR antibodies and peptide or chemical mimetics thereof, or FcR receptor ligands) and chemical moieties (e.g., dyes and synthetic FcR ligands). Such Fc receptor binding agents can be monospecific, bispecific or multispecific in that they contain one, two, or more than two binding regions, respectively. For example, the agent can bind to two or more different regions of an Fc receptor, or to an Fc receptor and a different component of the same or another cell. In all cases, the agent contains at least one portion which binds to an Fc receptor.

In one embodiment, the Fc receptor binding agent is an antibody, or an antibody fragment, including, e.g., an Fab, Fab′, F(ab′)₂, Fv, or a single chain Fv. The antibody may also be a light chain or heavy chain dimer, or any minimal fragment thereof such as a Fv, or a single chain construct as described in Ladner et al. U.S. Pat. No. 4,946,778, issued Aug. 7, 1990, the contents of which is expressly incorporated by reference.

In another embodiment, the Fc receptor binding agent is an antibody mimetic (e.g. peptide or chemical compound)(Jenks et al. J. Natl. Cancer Inst. (1992) 84(2):79; Saragovi et al. Science (1991) 253:792; Hinds et al. J. Med. Chem. (1991) 34:1777-1789; Fassina Immunomethods (1994) 5:121-129).

In another embodiment, the Fc binding component is a bispecific or a multispecific molecule. The term “bispecific molecule” is intended to include any compound, e.g., a chemical moiety or a protein, peptide, or protein or peptide complex, which has two different binding specificities which bind to, or interact with (a) an Fc receptor on the surface of a macrophage, and (b) a second, different target antigen. The term “multispecific molecule” or “heterospecific molecule” is intended to include any compound, e.g., a chemical moiety, a protein, peptide, or protein or peptide complex, which has more than two different binding specificities which bind to, or interact with (a) an Fc receptor on the surface of a macrophage, (b) two or more different target antigens. Accordingly, Fc receptor binding agents which can be used in macrophage-binding compounds of the invention include bispecific, trispecific, tetraspecific, and other multispecific molecules which are directed to Fc receptors on macrophages.

For example, the agent can be a heteroantibody comprising two or more antibodies, antibody binding fragments (e.g., Fab), or derivatives thereof, linked together which have different specificities. These different specificities can include two or more different binding specificities on an Fc receptor. Alternatively, they can include a binding specificity on an Fc receptor, and at least one other different binding specificity on the same cell (i.e., a macrophage) or on a different target cell (e.g., another immune cell or a pathogen).

In such embodiments where the Fc binding agent is a bispecific or multispecific molecule, the agent can function to physically bring together a cytotoxic effector cell to a target macrophage, such that more efficient, targeted elimination of the macrophage can be achieved. As used herein, the term “effector cell” refers to an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, eosinophils, neutrophils, polymorphonuclear cells, granulocytes, mast cells, and basophils. Like macrophages, effector cells express specific Fc receptors and carry out specific immune functions. In preferred embodiments, an effector cell is capable of inducing antibody-dependent cellular toxicity (ADCC), e.g., a neutrophil capable of inducing ADCC. For example, neutrophils, eosinophils, and lymphocytes which express FcαR are involved in specific killing of target cells and presenting antigens to other components of the immune system, or binding to cells that present antigens. In other embodiments, an effector cell can phagocytose a target antigen or cell (e.g., a macrophage), or microorganism, or can lyse a target cell, e.g., a macrophage. The expression of a particular Fc receptor on an effector cell can be regulated by humoral factors such as cytokines. For example, expression of FcγRI has been found to be up-regulated by interferon gamma (IFN-γ). This enhanced expression increases the cytotoxic activity of FcγRI-bearing cells against targets, e.g., macrophages.

In other embodiments of the invention, the Fc receptor binding agent is a monoclonal antibody or fragment thereof. The terms “monoclonal antibody” or “monoclonal antibody composition” as used herein refer to a preparation of antibody molecules of single molecular composition. A monoclonal antibody composition displays a single binding specificity and affinity for a particular epitope. The monoclonal antibody can be murine, or a human monoclonal antibody (e.g., an antibody produced in a mouse genetically-engineered to express completely human antibodies).

In still other embodiments of the invention, the Fc receptor binding agent is a chimeric antibody or fragment thereof, or a humanized antibody or fragment thereof. A “chimeric antibody” is intended to include an antibody in which the variable regions are from one species of animal and the constant regions are from another species of animal. For example, a chimeric antibody can be an antibody having variable regions which derive from a mouse monoclonal antibody and constant regions which are human. In a preferred embodiment of the invention, the macrophage-binding compound comprises a humanized antibody or binding fragment thereof. The term “humanized antibody” is intended to include antibodies in which the hypervariable regions, also termed, the complementarity-determining regions (CDRs) are from one species of animal and the framework regions and constant regions of the antibody are from a different species animal species. In a humanized antibody of the invention, the CDRs are from a mouse monoclonal antibody and the other regions of the antibody are human. In preferred embodiments, a human antibody is derived from known proteins NEWM and KOL for heavy chain variable regions (VHs) and REI for Ig kappa chain, variable regions (VKs). The term antibody as used herein is intended to include chimeric and humanized antibodies, binding fragments of antibodies or modified versions of such.

The terms “fragment” or “binding fragment” of an antibody or protein capable of binding to an antigen is intended to include a fragment of the antibody or protein which is sufficient for binding to the antigen. Binding of a binding fragment of an antibody to an antigen can be with the same affinity or a different affinity, e.g., lower or higher affinity, as binding of the whole antibody to the antigen. Examples of binding fragments encompassed within the term antibody include: an Fab fragment consisting of the V_{L}, V_{H}, C_{L} and C_{H1} domains; an Fd fragment consisting of the V_{H} and C_{H1} domains; an Fv fragment consisting of the V_{L} and V_{H} domains of a single arm of an antibody; a dAb fragment (Ward et al., 1989 Nature 341:544-546) consisting of a V_{H} domain; an isolated complementarity determining region (CDR); and an F(ab′)₂ fragment, a bivalent fragment comprising two Fab′ fragments linked by a disulfide bridge at the hinge region. A binding fragment, e.g., a binding fragment of an antibody, can be an active or functional binding fragment. Accordingly, an active or functional binding fragment is intended to include binding fragments which are capable of triggering at least one activity or function triggered by the full length molecule. For example, an active binding fragment of monoclonal antibody M22 or H22 is a fragment of the antibody that is capable of binding to the FcγR and triggering a receptor-mediated effector cell activity, e.g., production of superoxide anion. These antibody fragments are obtained using conventional techniques known to those with skill in the art, and the fragments are screened for utility in the same manner as are intact antibodies.

The terms “an agent which binds to” or “binding specificity” is used interchangeably herein with the terms “antigen binding site, “antigen binding region” and “binding determinant of an antibody.” These terms are intended to include the region of a molecule, e.g., an antibody, that are involved in the binding to an antigen. The antigen binding site of an antibody comprises, but is not limited to, the amino acids of the antibody which contact the antigen. The antigen binding region can be the variable region of an antibody. The antigen binding region of an antibody can also be the hypervariable regions of an antibody. The antigen binding region of an antibody can also be the amino acid residues in the hypervariable region of an antibody which contact the antigen and/or which provide proper tertiary structure of the antigen binding region. Various methods are available for determining which amino acid residues of a variable region or hyper variable region of an antibody contact the antigen and/or are important in having a correctly folded antigen binding region. For example, mutagenesis analyses can be performed. In particular, it is possible to substitute one or more amino acids for other amino acids in a recombinantly produced antibody and to perform in vitro binding studies to determine the extent to which the binding affinity of the modified antibody for the antigen has changed compared to the non modified antibody. If binding has decreased due to substitution of an amino acid for another, the amino acid is most likely important in binding of the antibody to the antigen. Other methods for determining which amino acids of a variable region of an antibody are involved in binding of the antibody to an antigen are based on crystallographic analyses, e.g., X-ray crystallography.

The term “an antibody which binds specifically to an antigen” is intended to include an antibody which binds to the specific antigen with significantly higher affinity than binding to any other antigen, i.e., it is intended to define the specificity of an antibody as defined in the art. The terms “an antibody recognizing an antigen” and “an antibody specific for an antigen” are used interchangeably herein with the term “an antibody which binds specifically to an antigen”.

Production of Anti-Fc Receptor Binding Agents

I. Production of Anti-Fc Receptor Antibodies

Anti-Fc receptor antibodies for use in macrophage-binding compounds of the invention include antibodies developed using any of a variety of known techniques, provided that the antibody is capable of binding to an Fc receptor on a macrophage. Preferred antibodies are practical for clinical use (e.g., can be administered to humans). Particularly preferred antibodies are non-immunogenic when administered to humans (e.g., are human antibodies produced in transgenic animals), or are modified to reduce immunogenicity when administered to humans (e.g., are humanized).

In one embodiment, the anti-Fc receptor antibody is a monoclonal antibody, e.g., a murine or human monoclonal antibody, which binds to a type IgG receptor or a type IgA receptor, preferably at a site which is not blocked (i.e., bound) by human immunoglobulin G (IgG) or immunoglobulin A (IgA). As used herein, the term “IgG receptor” refers to any of the eight Fcγ receptor genes located on chromosome 1. These genes encode a total of twelve transmembrane or soluble receptor isoforms which are grouped into three Fcγ receptor classes: FcγRI (CD64), FcγRII(CD32), and FcγRIII (CD 16). In one preferred embodiment, the Fcγ receptor is a human high affinity FcγRI. The human FcγRI is a 72 kDa molecule, which shows high affinity for monomeric IgG (10⁸-10⁹M⁻¹). The production and characterization of these preferred monoclonal antibodies are described by Fanger et al. in PCT application WO 88/00052 and in U.S. Pat. No. 4,954,617, the teachings of which are fully incorporated by reference herein. These antibodies bind to an epitope of FcγRI, FcγRII or FcγRIII at a site which is distinct from the Fcγ binding site of the receptor and, thus, their binding is not blocked substantially by physiological levels of IgG. Specific anti-FcγRI antibodies useful in this invention are mAb 22, mAb 32, mAb 44, mAb 62 and mAb 197. The hybridoma producing mAb 32 is available from the American Type Culture Collection, ATCC Accession No. HB9469. Anti-FcγRI mAb 22, F(ab′)₂ fragments of mAb 22, and can be obtained from Medarex, Inc. (Annandale, N.J.). The hybridoma producing MAb 22 is available from the ATCC on Jul. 9, 1996 and has been assigned ATCC Accession No. HB-12147. In other embodiments, the anti-Fcγ receptor antibody is a humanized form of monoclonal antibody 22 (H22). The production and characterization of the H22 antibody is described in Graziano, R. F. et al. (1995) J. Immunol 155 (10): 4996-5002 and PCT/US93/10384. The H22 antibody producing cell line was deposited at the American Type Culture Collection on Nov. 4, 1992 under the designation HA022CL1 and has the accession no. CRL 11177.

In other embodiments, the anti-FcR antibody is specific for an IgA receptor. The term “IgA receptor” is intended to include the gene product of one α-gene (FcαR) located on chromosome 19. This gene is known to encode several alternatively spliced transmembrane isoforms of 55 to 110 kDa. FcαR (CD89) is constitutively expressed on monocytes/macrophages, eosinophilic and neutrophilic granulocytes, but not on non-effector cell populations. FcαR has medium affinity (≈5×10⁷ M⁻¹) for both IgA1 and IgA2, which is increased upon exposure to cytokines such as G-CSF or GM-CSF (Morton, H. C. et al. (1996)Critical Reviews in Immunology 16:423-440). Exemplary anti-Fcα receptor monoclonal antibodies include My 43, A77, A62, A59, and A3 (Monteiro et al. (1992) J. Immunol. 148:1764; Shen et al. (1989) J. Immunol. 143: 4117). Preferred anti-FcαR antibodies are capable of binding to an FcαR without being inhibited by IgA. The antibody A77 has been produced by immunizing mice with acrylamide gel slices containing FcαR that was IgA affinity purified from human cell lysates. Monoclonal antibodies were screened according to three characteristics: staining of U937 cells at a higher density after PMA activation, selective reactivity with blood monocytes and granulocytes, and their ability to immunoprecipitate molecules of approximately 55 to 75 kDa from neutrophils and activated U937 cells.

Monoclonal anti-Fc receptor antibodies used in the compounds of the invention can be produced by a variety of techniques, including conventional monoclonal antibody methodology, e.g., the standard somatic cell hybridization technique of Kohler and Milstein, (1975) Nature 256: 495. Although somatic cell hybridization procedures are preferred, in principle, other techniques for producing monoclonal antibody can be employed e.g., viral or oncogenic transformation of B lymphocytes.

A preferred animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known.

Human monoclonal antibodies (mAbs) directed against human proteins can be generated using transgenic mice carrying the complete human immune system rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. (1994) Proc. Natl. Acad. Sci. USA 81:6851-6855; Bruggeman et al. (1993) Year Immunol 7:33-40; Tuaillon et al. (1993) PNAS 90:3720-3724; Bruggeman et al. (1991) Eur. J. Immunol. 21:1323-1326).

In an illustrative embodiment, mice (HuMab mice) which produce a fully human antibody response after immunization can be generated by inactivating the genes coding for mouse antibodies. This can be achieved by generating a ‘double-knockout mouse’ in which the endogenous immunoglobulin heavy chain and the κ-light chain genes are disrupted by targeted deletion of the exons coding for the constant regions (Cμ and JCκ). Separate transgenes can be constructed which contain both the human immunoglobulin heavy chain genes and the human κ light chain genes. In humans, these genes encompass about 1-2 megabases each, a size which is too large to isolate intact. The essential regions can be assembled in condensed form in so-called ‘miniloci’. The heavy chain minilocus contains 2-6 Vₕ gene segments, 15 Dₕ and 6 Jₕ gene segments, and the Sμ and Cμ and Sγ1 and Cγ1 gene segments. The κ-light chain minilocus contains 1-17 Vκ-gene segments, 5 Jκ and the Cκ gene segments (Lonberg, N. et al. (1994) Nature 368: 856-859; Tuaillon, N. et al. (1993) Proc. Natl. Acad. Sci. USA 90: 3720-3724). These miniloci can be subsequently incorporated into the genome of the ‘double-knockout’ mice. Several consecutive versions of these double-knockout/double transgenic HuMab mice can be generated, which incorporate increasing amounts of the human heavy- and light-chain loci. For example, HuMab mice have been generated which incorporate a 100 kb heavy chain transgene containing six V segments, and a 200 kb κ light chain transgene containing 17 Vκ-segments. These HuMab mice can be immunized using conventional immunization protocols, and have been shown to efficiently generate high-affinity human IgG1 antibodies against a broad panel of antigens (Fishwild, D. M. et al. (1996) Nature Biotech 14: 845-851; Lonberg, N. and D. Huszar (1995) Int. Rev. Immunol. 13: 65-93). The antibodies generated following these protocols have been shown to have excellent biological activity, and long serum half-lifes.

Chimeric mouse-human monoclonal antibodies (i.e., chimeric antibodies) can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted. (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

The chimeric antibody can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from 7E3, an anti-GPII_{b}IIIₐ antibody producing hybridoma. The recombinant DNA encoding the chimeric antibody, or fragment thereof, can then be cloned into an appropriate expression vector. Suitable humanized antibodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060.

All of the CDRs of a particular human antibody may be replaced with at least a portion of a non-human CDR or only some of the CDRs may be replaced with non-human CDRs. It is only necessary to replace the number of CDRs required for binding of the humanized antibody to the Fc receptor.

An antibody can be humanized by any method, which is capable of replacing at least a portion of a CDR of a human antibody with a CDR derived from a non-human antibody. Winter describes a method which may be used to prepare the humanized antibodies of the present invention (UK Patent Application GB 2188638A, filed on Mar. 26, 1987), the contents of which is expressly incorporated by reference. The human CDRs may be replaced with non-human CDRs using oligonucleotide site-directed mutagenesis as described in International Application WO 94/10332 entitled, Humanized Antibodies to Fc Receptors for Immunoglobulin G on Human Mononuclear Phagocytes.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances. Antibodies in which amino acids have been added, deleted, or subsituted are referred to herein as modified antibodies or altered antibodies.

The term modified antibody is also intended to include antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody. Any modification is within the scope of the invention so long as the macrophage-binding compound has at least one antigen binding region specific for an FcR and triggers at least one effector function.

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies (for descriptions of combinatorial antibody display (see e.g., Sastry et al. (1989) PNAS 86:5728; Huse et al. (1989) Science 246:1275; and Orlandi et al. (1989) PNAS 86:3833). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR. For instance, mixed oligonucleotide primers corresponding to the 5′ leader (signal peptide) sequences and/or framework 1 (FR1) sequences, as well as primer to a conserved 3′ constant region primer can be used for PCR amplification of the heavy and light chain variable regions from a number of murine antibodies (Larrick et al., 1991, Biotechniques 11: 152-156). A similar strategy can also been used to amplify human heavy and light chain variable regions from human antibodies (Larrick et al., 1991, Methods: Companion to Methods in Enzymology 2:106-110).

In an illustrative embodiment, RNA is isolated from B lymphocytes, for example, peripheral blood cells, bone marrow, or spleen preparations, using standard protocols (e.g., U.S. Pat. No. 4,683,202; Orlandi, et al. PNAS (1989) 86:3833-3837; Sastry et al., PNAS (1989) 86:5728-5732; and Huse et al. (1989) Science 246:1275-1281.) First-strand cDNA is synthesized using primers specific for the constant region of the heavy chain(s) and each of the κ and λ light chains, as well as primers for the signal sequence. Using variable region PCR primers, the variable regions of both heavy and light chains are amplified, each alone or in combination, and ligated into appropriate vectors for further manipulation in generating the display packages. Oligonucleotide primers useful in amplification protocols may be unique or degenerate or incorporate inosine at degenerate positions. Restriction endonuclease recognition sequences may also be incorporated into the primers to allow for the cloning of the amplified fragment into a vector in a predetermined reading frame for expression.

The V-gene library cloned from the immunization-derived antibody repertoire can be expressed by a population of display packages, preferably derived from filamentous phage, to form an antibody display library. Ideally, the display package comprises a system that allows the sampling of very large variegated antibody display libraries, rapid sorting after each affinity separation round, and easy isolation of the antibody gene from purified display packages. In addition to commercially available kits for generating phage display libraries (e.g., the Pharmacia Recombinant Phage Antibody System, catalog no. 27-9400-01; and the Stratagene SurZAP™ phage display kit, catalog no. 240612), examples of methods and reagents particularly amenable for use in generating a variegated antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982.

In certain embodiments, the V region domains of heavy and light chains can be expressed on the same polypeptide, joined by a flexible linker to form a single-chain Fv fragment, and the scFV gene subsequently cloned into the desired expression vector or phage genome. As generally described in McCafferty et al., Nature (1990) 348:552-554, complete V_{H} and V_{L} domains of an antibody, joined by a flexible (Gly₄-Ser)₃ linker can be used to produce a single chain antibody which can render the display package separable based on antigen affinity. Isolated scFV antibodies immunoreactive with the antigen can subsequently be formulated into a pharmaceutical preparation for use in the subject method.

Once displayed on the surface of a display package (e.g., filamentous phage), the antibody library is screened with the FcγR, or peptide fragment thereof, to identify and isolate packages that express an antibody having specificity for the FcγR. Nucleic acid encoding the selected antibody can be recovered from the display package (e.g., from the phage genome) and subcloned into other expression vectors by standard recombinant DNA techniques.

Anti-Fc receptor binding agents, and/or other binding agents within macrophage-binding compounds of the invention with high affinities for a target antigen (e.g., surface protein) can be made according to methods known to those in the art, e.g, methods involving screening of libraries (Ladner, R. C., et al., U.S. Pat. No. 5,233,409; Ladner, R. C., et al., U.S. Pat. No. 5,403,484). Further, the methods of these libraries can be used in screens to obtain binding determinants that are mimetics of the structural determinants of antibodies. In particular, the Fv binding surface of a particular antibody molecule interacts with its epitope according to principles of protein-protein interactions, hence sequence data for V_{H} and V_{L} (the latter of which may be of the κ or λ chain type) is the basis for protein engineering techniques known to those with skill in the art. Details of the protein surface that comprises the binding determinants can be obtained from antibody sequence information, by a modeling procedure using previously determined three-dimensional structures from other antibodies obtained from NMR studies or crytallographic data. See for example Bajorath, J. and S. Sheriff, 1996, Proteins: Struct., Funct., and Genet. 24 (2), 152-157; Webster, D. M. and A. R. Rees, 1995, “Molecular modeling of antibody-combining sites,” in S. Paul, Ed., Methods in Molecular Biol. 51, Antibody Engineering Protocols, Humana Press, Totowa, N.J., pp 17-49; and Johnson, G., Wu, T. T. and E. A. Kabat, 1995, “Seqhunt: A program to screen aligned nucleotide and amino acid sequences,” in Methods in Molecular Biol. 51, op. cit., pp 1-15.

In one embodiment, the anti-Fc receptor binding agent includes an antigen binding site that is derived from an antibody and which is grafted onto a non-antibody molecule. For example, an antigen binding region can be grafted onto a peptide or protein. In one embodiment, one portion of the antigen binding region, e.g., the portion similar to the antigen binding region from the light chain of an antibody, is grafted onto one protein or peptide and the other portion of the antigen binding region, e.g., the portion similar to the antigen binding region from the heavy chain of an antibody, is grafted onto another protein or peptide. In a preferred embodiment of the invention, the two proteins or peptides having each a portion of the antigen binding region are linked, e.g., by chemical linkage, recombinantly, or by non covalent interaction, such as to produce a protein having an antigen binding site specific for an FcR for human Igs, which triggers at least one Fc receptor-mediated effector cell function.

An antigen binding region can also be obtained by screening various types of combinatorial libraries with a desired binding activity, and to identify the active species, by methods that have been described. For example, phage display techniques (Marks et al. (1992) J Biol Chem 267:16007-16010) can be used to identify proteins binding FcγRs. Phage display libraries have been described above. For example, a variegated peptide library can be expressed by a population of display packages to form a peptide display library. Ideally, the display package comprises a system that allows the sampling of very large variegated peptide display libraries, rapid sorting after each affinity separation round, and easy isolation of the peptide-encoding gene from purified display packages. Peptide display libraries can be in, e.g., prokaryotic organisms and viruses, which can be amplified quickly, are relatively easy to manipulate, and which allows the creation of large number of clones. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages. Phage display libraries are described above.

Other techniques include affinity chromatography with an appropriate “receptor”, e.g., FcγRI or FcαR, to isolate binding agents, followed by identification of the isolated binding agents or ligands by conventional techniques (e.g., mass spectrometry and NMR). Preferably, the soluble receptor is conjugated to a label (e.g., fluorophores, calorimetric enzymes, radioisotopes, or luminescent compounds) that can be detected to indicate ligand binding. Alternatively, immobilized compounds can be selectively released and allowed to diffuse through a membrane to interact with a receptor.

Combinatorial libraries of compounds can also be synthesized with “tags” to encode the identity of each member of the library (see e.g., W. C. Still et al., International Application WO 94/08051). In general, this method features the use of inert but readily detectable tags, that are attached to the solid support or to the compounds. When an active compound is detected, the identity of the compound is determined by identification of the unique accompanying tag. This tagging method permits the synthesis of large libraries of compounds which can be identified at very low levels among to total set of all compounds in the library.

II. Cyanin Compositions

In another embodiment of the invention, the Fc receptor binding agent of the macrophage-binding compound is a chemical moiety, such as a cyanin composition, including but not limited to the fluorescent dye Cy5.18.OSu (referred to as Cy5) and conjugates and derivatives thereof. Cyanin compositions are known to bind with high affinity and specificity to FcγRI receptors. In certain cases, the cyanin compositions can contain two or more moieties, such as a cyanin succinimidyl ester and a phycobilisome protein, e.g., PE. The term “PE-Cy5” as used here designates the specific tandem dye comprised of phycoerythrin and Cy5.18.OSu; the term “PE-Cy5 reagent” designates, for example but not limited to, PE-Cy5 conjugates to antibodies, to genetically engineered binding proteins and peptides (U.S. Pat. Nos. 5,233,409 and 5,403,484), to avidin, to biotin, or to other molecular entities. PE-Cy5 conjugates can be used in therapeutic and diagnostic applications.

Cyanin was isolated from cornflower (Centaurea cyanus), and is structurally the 3,5-diglucoside of cyanidin, which is 2-(3,4-dihydroxyphenyl)-3,5,7-trihydroxy-1-benzopyrylium chloride and was isolated from banana (Merck Index). Another cyanidin derivative, the 3-rhamnoglucoside isolated from sour cherries, is described as having therapeutic application for night blindness. Anthocyanosides of bilberry (Vaccinium myrtillus) fruit are marketed as nutraceutical food supplements, which according to one manufacturer (Amrion, Inc., Boulder, Colo.), are consumed orally to improve vasodilation, decrease capillary permeability, protect collagen in blood vessels, operate as antioxidants and support control of the inflammatory process, improving general vision, stomach linings, blood-brain barrier and the veins of the legs and colon (Gen. Engin. News 16 (11), p.27, 1996).

The cyanidin derivative dye Cy5, also designated Cy5.18.OSu, has the chemical structure 5,5′-bis-sulfo-1,1′-(ε-carboxyphenyl)-3,3,3′,3′-tetramethylindodicarbocyanin-disuccinimidyl ester (A. S. Waggoner et al., In: Clinical Flow Cytometry, p.185 (Eds) A. Landay et al. The New York Academy of Sciences, New York, N.Y., 1993). Cyanin dye labeling reagents for sulfhydryl groups (Ernst, L. A. et al., 1989, Cytometry 10:3) and carboxymethylindocyanin succinimidyl esters (Southwick, P. L. et al., 1990, Cytometry 11:418) have been described, and compositions claimed in patent applications (U.S. Pat. Nos. 4,981,977 and 5,268,486), the contents of which are hereby incorporated by reference. Structure of Cy5, and its synthesis and spectra for absorption and emission of light are given in Mujumdar, R. B., 1993, Bioconj. Chem. 4:105. Cy5 is a sulfoindocyanin succinimidyl ester, which is an amino-reactive cyanin dye that contains a negatively charged sulfonate group on the aromatic nucleus of the indocyanin fluorophore. The Cy5 members of this family are characterized by a 5-carbon, unsaturated polymethine bridge connecting two substituted ring structures. Cy5 can be excited with a 633 nm HeNe laser line or a 647 nm line of a Dr laser. Cy5 and its derivatives are noted for photostability, which is comparable to or better than that of fluorescein. The extinction coefficient (L/mol cm) of 250,000 is very high. Related dyes (Mujumdar et al., supra), with similar structures and modes of synthesis are here encompassed within the expression “Cy5” so that this expression encompasses sulfoindocyanin succinimidyl esters of cyanin dye labeling reagents in general, for example, Cy3.29.OSu (known as Cy3) and Cy7.18.OH. The terms Cy5 reagent, Cy5 conjugate and Cy5 derivatives shall mean a conjugate comprising at least a Cy5 moiety and another molecular entity. Additional new derivatives of this basic structure have been described, the sulfobenzindocyanin succinimidyl esters of cyanin reagents (Mujumdar, S. R. et al., 1996, Bioconj. Chem 7:356), which share properties of Cy5 and other sulfoindocyanin succinimidyl esters, and are contemplated to bind FcγRI with affinity and specificity.

Use of the Cy5 reagent PE-Cy5, comprised of Cy5 in tandem with PE, to provide three-color fluorescence by excitation with a single 488 nm argon ion laser line is described in Waggoner et al., 1993, supra, as are conditions for optimization. Major problems with tandem dyes based on Texas Red are attributed to instability of one moiety, resulting during use in leakage of emission into the spectrum of the other moiety, limiting the ability to use Texas Red dyes emitting light at or near the wavelength of that second moiety. Cy5 and its reagent family of dyes, however, emit light at longer wavelengths than Texas Red, so that analysis of data obtained from using Cy5 with other dyes requires minimal channel compensation in setting detection windows and in downstream calculations. Considerations for best mode use of Cy5 reagents include the process of synthesis of the Cy5 reagent from the components, since the ratio of number of Cy5 molecules bound per molecule of conjugate affects the relative emission wavelength spectrum of the synthesis product. Thus for PE-Cy5, the efficiency of energy transfer from PE to Cy5 increases as more Cy5 molecules are bound to each PE up to an optimal range, beyond which quenching interactions among excess Cy5 moieties is observed. The optimum ratio is 4 to 8 Cy5 per PE in the PE-Cy5 tandem dye (Waggoner et al., 1993, supra). Tandem dyes are light sensitive, and stability during usage is improved if dyes are stored and handled and experiments are performed under dark conditions.

The improved signal size due to extent of fluorescence and absence of background for PE-Cy5, compared to that of previously synthesized tandem dyes, make it a successful analytical tool for cell analysis studies with antibody-dye conjugates. However at least one report of “non-specific” binding of a variety of PE-Cy5 products from different suppliers to myeloid cells has been reported (Stewart S J, et al., supra), attributed to the Cy5 moiety because PE-Texas Red conjugates do not exhibit this property. In contrast, Takizawa et al. report binding of PE and its mAb conjugates to low affinity mouse IgG receptors FcγRII and FcγRIII (J. Immunol. Methods, 1993, 162:269).

Production of Cytotoxic Agents Which Kill Macrophages or Which Reduce Their Activity

I. Cytotoxins

A variety of cytotoxic agents can be targeted to macrophages via compounds of the invention (i.e., by virtue of being linked to an agent which binds to an Fc receptor on a macrophage). As used herein, the terms “cytotoxin” and “cytotoxic agent” includes any compound (e.g., drug) capable of killing or reducing the activity of a macrophage. For example, the compound can be a toxin, such as Gelonin, Saporin, Exotoxin A, Onconase or Ricin A, or a drug, such as dichloromethylene diphosphonate (CL2MDP) or a derivative thereof. Cytotoxins for use in the invention can additionally include an agent or a moiety which enhances the therapeutic activity of these compounds.

For example, the cytotoxin can include an agent which promotes apoptosis, a mitotic inhibitor, an alkylating agent, an antimetabolite, a nucleic acid intercalating agent, a topoisomerase inhibitor, a macrophage-specific drug, or a radionuclide. The present invention offers the advantage of targeting such cytotoxins to high affinity Fcγ receptors (e.g., using an antibody such as Mab 22, Mab 32, or humanized forms thereof) on macrophages where they, for example, are internalized by the cell. Therefore, these cytotoxins can be more effective in cell killing or modulating cell function than other agents which are not internalized, or that are internalized with slower kinetics.

The cytoxic agent can be a toxic drug or an enzymatically active toxin of bacterial or plant origin, or a biologically active fragment (“A chain”) of such a toxin. Exemplary enzymatically active toxins and fragments thereof include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthinproteins, phytolacca americana proteins (PAPI, PAPII, and PAP-S), momordicacharantia inhibitor, curcin, crotin, saponaria officinalis inhibitor, gelonin, mitogellin, restrictocin, phenomycin and enomycin. Preferred toxins that can be used include Gelonin, Saporin, Exotoxin A, Onconase, Ricin A, diphtheria toxin, and Pseudomonas exotoxin or subunits of these toxins. Studies the preparation, in vivo uses and pharmacokinetics of these toxins are described in, for example, Vitetta et al. (1987) Science 238: 1098-1104; Spitlet, L. et al. (1987) Clin. Chem. 33(b): 1054; Uhr et al., Monoclonal Antibodies and Cancer, Academic Press, Inc., pp. 85-98 (1983). Conjugates of the compounds of the invention and such toxic agents may be prepared using a variety of bifunctional protein coupling agents as described in detail below in the section entitled “Methods of Making Conjugates of Macrophage-Binding Compounds.” Examples of such reagents are SPDP, IT, bifunctional derivatives of imidoesters such as dimethyl adipimidate, HC1, active esters such as disuccinimidyl suberate, aldehydes such as glutaraldehyde, bis-azido compounds such as bis-(p-azidobenzoyl) hexanediamine, bis-diazonium derivatives such as bis-(p-diazoniumbenzoyl)-ethylenediamine, diisocyanates such as tolulene 2,6-diisocyanate, and bis-active fluorine compounds such as 1,5-difluoro-2,4-dinitrobenzene.

In other embodiments, the cytotoxin is a drug. Exemplary drugs include dichloromethylene diphosphonate (CL2MDP) or other chlodronate derivatives (Bogers et al. (1991) Clin. Exp. Immunol. 86: 328-333). Alternatively, the cytotoxin can be an agent which promotes apoptosis, a mitotic inhibitor, an alkylating agent, an antimetabolite, a nucleic acid intercalating agent, and a topoisomerase inhibitor. Examples of such agents which can be used in the compounds of the invention include the topoisomerase II inhibitors ellipticine, amsacrine, adriamycin and mitrozantrone, the prokaryotic DNA gyrase inhibitor coumermycin A1 and the DNA binding agents neocarzinostatin and chloroquine (which either intercalate or nick DNA). Methods for delivery of such drugs, e.g., liposome-delivery, are described below.

In certain embodiments, the cytotoxin can comprise a photosensitizing moiety (e.g., a photosensitizing drug). Cytotoxins which constitute such photosensitizing moieties are useful in sensitizing a target, e.g., a macrophage, to destruction upon photoactivation, e.g., by irradiation using visible light. Preferably, the photosensitizing moiety has no direct biological effect prior to photoactivation. Compounds comprising such moieties can be administered to a subject, e.g., topically or by injection. Upon photoactivation by exposing these compounds to a particular wavelength of light, e.g., by visible light exposure, the moiety becomes toxic (either itself or by activating a cytotoxin associated with the moiety) and selectively destroys the macrophages. Without being bound by any particular theory, the mechanism of photoactivation is believed to include transfer of energy from a photosensitizing moiety to endogenous oxygen, thereby converting it to singlet oxygen. The singlet oxygen is thought to be responsible for the cytotoxic effect. Macrophage binding compounds containing photosensitizing moieties are particularly useful for treatment of dermatological diseases.

Exemplary photosensitizing agents that can be used in the present invention include porphyrin related compounds, e.g. hematoporphyrin derivatives (Lipson, R. L. et al. (1961), J. National Cancer Inst. 26:1-8; Photophrin II compositions (U.S. Pat. No. 4,649,151, Dougherty, T. J. (1983) Adv. Exp. Med. Bio. 160: 3-13, Kessel, D. et al. (1987) Photochem. Photobiol. 46: 463-568 and Scourides, P. A. et al. (1987) Cancer Res. 47: 3439-3445), pyropheophorbide compounds (U.S. Pat. No. 5,459,159; U.S. Pat. No. 4,996,312, and U.S. Pat. No. 4,849,207, and EP 220686); chlorophyll and bacteriophyll derivatives (EPA 93111942.4); 9-substituted porphycene derivatives (WO 96/31451); phrobine derivatives (WO 95/08551); as well as chlorins, phthalocyanines and porphins (reviewed in Harvey, I. Pass. (1993) J. Natl. Canc. Inst. 85: 443-457). Photoactivated forms of photosensitizing agent which are capable of emitting a fluorescent signal can also be used in diagnostic applications to label macrophage-binding compounds of the invention.

In other embodiments, the macrophage binding compounds of the invention can include an Fc binding agent coupled to a therapeutic or a diagnostic reagent, e.g., toxic agent, via a photocleavable linkage. Preferably, the linkage is mediated by a photoactivable agent, such as a chromophore, which releases the therapeutic or diagnostic reagent upon exposure to light (Goldmacher et al. (1992) Bioconj. Chem. 3: 104-107). For example, in dermatological applications, light will induce degradation of the linkage, liberating the active toxin locally (e.g., skin). Photoactivatable agents suitable for releasing the bound therapeutic or diagnostic reagent include any agent which can be linked to a functional group (e.g., a phenol) of the therapeutic or diagnostic reagent and which, upon exposure to light, releases the therapeutic or diagnostic reagent in functional form. As an illustration, the photoactivatable agent can be a chromophore. Suitable chromophores are generally selected for absorption of light that is deliverable from common radiation sources (e.g. UV light ranging from 240-370 nm). Examples of chromophores which are photoresponsive to such wavelengths include, but are not limited to, acridines, nitroaromatics and arylsulfonamides.

When using chromophores, the efficiency and wavelength at which the chromophore becomes photoactivated and thus releases or “uncages” the therapeutic reagent will vary depending on the particular functional group(s) attached to the chromophore. For example, when using nitroaromatics, such as derivatives of o-nitrobenzylic compounds, the absorption wavelength can be significantly lengthened by addition of methoxy groups. In one embodiment, nitrobenzyl (NB) and nitrophenylethyl (NPE) is modified by addition of two methoxy residues into 4,5-dimethoxy-2-nitrobenzyl (DMNB) and 1-(4,5-dimethoxy-2-nitrophenyl)ethyl (DMNPE), respectively, thereby increasing the absorption wavelength range to 340-360 nm (λₘₐₓ=355 nm). Radiation to promote photorelease of the therapeutic or diagnostic agent can be provided by a variety of sources including, but not limited to, non-coherent UV light sources and excimer sources. In one embodiment, a KrF excimer laser operating at 248 nanometers can be used. Alternatively, a frequency-quadrupled, solid state, Neodymium-doped YAG laser or the like operating at 266 nm can be used, or an Argon ion laser operating at 257 or 275 nm can be used. The photoactivatable agent can be reacted with the therapeutic agent to create a photoreleasable linkage. When using chromophores as photoactivatable agents, the excitation wavelength may be chosen so as to selectively excite particular chromophores. For example, it is possible to photoreleasably attach two different drugs or to two different chromophores to the substrate, and then independently or sequentially release the two drugs by selecting the excitation wavelength to match the corresponding chromophore. The chromophore and the excitation wavelength may further be selected to avoid undesired photolytic reactions of the drug (e.g., inactivation) or of the surrounding tissue. For example, the photosensitivity of nucleic acids is well known. When the drug is a nucleic acid, excitation energy which may damage the nucleic acid (e.g. wavelengths shorter than 280 nm) should be avoided.

In addition, macrophage-binding compounds of the invention can be labeled (e.g., for diagnostic use) by coupling the compound to radionuclides, such as 131I, 90Y, 105Rh, 47Sc, 67Cu, 212Bi and 211At, as described, e.g., in Goldenberg, D. M. et al. (1981) Cancer Res. 41: 4354-4360; in EP 0365 997; Carrasquillo et al., Cancer Treat. Rep., 68:317-328 (1984); Zaloberg et al., J. Natl. Cancer Institute 72:697-704 (1984); Jones et al., Int. J. Cancer 35:715-720 (1985); Lange et al., Surgery 98:143-150 (1985); Kaltovich et al., J. Nucl. Med. 27:897 (1986); Order et al., Intl. J. Radiother. Oncl. Biol. Phys. 8:259-261 (1982); Courtenay-Luck et al. Lancet 1:1441-1443 (1983); Ettinger et al., Cancer Treat. Rep. 56:289-297 (1982); the disclosures of all of which are incorporated herein by reference. Such radionuclides can also enhance the cytotoxic effect of the photosensitizing moiety.

In such diagnostic applications, it is desirable to attach a label group to the macrophage-binding compounds to facilitate their detection (e.g., their binding to macrophages in a sample). Accordingly, in addition to the radionuclides listed above, suitable labeling groups include, for example, a fluorophore, a colorimetric enzyme, a radioisotope, or a luminescent compound. For example, when the labeling group is an enzyme, the enzyme which is linked to the macrophage binding compound will react with an appropriate substrate, preferably a chromogenic substrate, in such a manner as to produce a chemical signal which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection of binding of macrophage-binding compounds to macrophages can also be accomplished using any of a variety of immunoassays. For example, a radioimmunoassay (RIA) can be used (see, for example, Weintraub, B., Principles of Radioimmunoassays, Seventh Training Course on Radioligand Assay Techniques, The Endocrine Society, March, 1986, which is incorporated by reference herein). Alternatively, enzyme immunoassays (EIA) can be used (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The radioactive isotope can be detected by such means as the use of a y counter or a scintillation counter or by autoradiography.

It is also possible to label the macrophage-binding compounds with a fluorescent compound. When the fluorescently labeled compound is exposed to light of the proper wavelength, its presence can then be detected. Among the most commonly used fluorescent labeling compounds are fluorescein isothiocyanate, rhodamine, phycoerythrin, phycocyanin, allophycocyanin, o-phthaldehyde and fluorescamine.

The compounds of the present invention can also be labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA). Alternatively, these compounds can be labeled by coupling them to a chemiluminescent compound. The presence of the chemiluminescent-tagged compound is then determined by detecting luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the macrophage-binding compounds of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

conjugating Anti-Fc Receptor Binding Agents to Cytotoxins

Macrophage-binding compounds of the present invention contain, along with other optional components, an agent which binds to an Fc receptor on a macrophage linked to a cytotoxin. Accordingly, to produce such compounds, the anti-Fc receptor binding agent is conjugated (e.g., by covalently crosslinking) to a cytotoxin using a variety of known techniques (see e.g., D. M. Kranz et al. (1981) Proc. Natl. Acad. Sci. USA 78:5807, U.S. Pat. No. 4,474,893), or by recombinantly expressing the anti-Fc receptor binding agent and the cytotoxin together as a fusion molecule.

Suitable agents, such as crosslinking agents, which can be employed for this purpose are well known in the art. The terms “crosslinking agent” and “crosslinker” are intended to include molecules which can function as bridging molecules between two other molecules by way of having two reactive functional groups, one of which reacts to form a covalent bond with the first molecule and the other of which reacts to form a covalent bond with the second molecule, thereby effectively connecting the two molecules together. Preferably, the crosslinker has two reactive functional groups of different functional moieties. Examples of suitable functional groups include amino groups, carboxyl groups, sulfhydryl groups and hydroxy groups. When one functional group of the crosslinker is reacted with a molecule (e.g., an Fc receptor binding agent), the other functional group can be, if necessary, prevented from reacting with that molecule by means of a protecting group which modifies the second functional group of the crosslinker so that it cannot react with the molecule. After the first reaction is completed, the protecting group can be removed, restoring the second functional group, and then the second functional group can be reacted with another molecule (e.g., a toxin).

Macrophage-binding compounds of the present invention can be prepared by conjugating their constituent agents, e.g., the anti-FcR and cytotoxin, using methods known in the art. For example, each agent of the macrophage-binding compound can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described by Paulus (Behring Ins. Mitt. (1985) No. 78, 118-132); Brennan et al. (Science (1985) 229:81-83), and Glennie et al. (J. Immunol. (1987) 139: 2367-2375). Preferred conjugating agents are SATA and sulfo-SMCC, both available from Pierce Chemical Co. (Rockford, Ill.).

In cases where the macrophage-binding molecule contains two antibodies (e.g., a bispecific antibody), these antibodies can be conjugated via sulfhydryl bonding of the C-terminus hinge regions of the two heavy chains. In a particularly preferred embodiment, the hinge region is modified to contain an odd number of sulfhydryl residues, preferably one, prior to conjugation. Alternatively, both agents can be encoded in the same vector and expressed and assembled in the same host cell. This method is particularly useful where the macrophage-binding compound is a mAb×mAb, mAb×Fab, Fab×F(ab′)₂ or ligand×Fab fusion protein. A macrophage-binding compound of the invention, e.g., a bispecific molecule can be a single chain molecule, such as a single chain bispecific antibody, a single chain bispecific molecule comprising one single chain antibody and a binding determinant, or a single chain bispecific molecule comprising two binding determinants. Macrophage-binding compounds can also be single chain molecules or may comprise at least two single chain molecules. Methods for preparing bi- and multispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Once produced in accordance with the guidelines above, macrophage-binding compounds can be tested for binding to macrophages using known techniques, such as enzyme-linked immunosorbent assay (ELISA), radioimmunoassay (RIA), or Western Blot Assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

Pharmaceutical Compositions and Administration Routes

Macrophage-binding compounds of the invention are preferably present in a composition along with a carrier or diluent. For in vivo administration to a subject (e.g., to treat or diagnose a disorder), the compounds are preferably present along with a pharmaceutically acceptable carrier or diluent. As described in detail below, pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

Pharmaceutical compositions of the invention also can be administered in a combination therapy, i.e., combined with other agents. For example, the combination therapy can include a composition of the present invention with at least one other anti-macrophage agent, or other conventional therapy. Exemplary anti-macrophage agents include chlodronate compounds, e.g., dichloromethylene diphosphonate (CL2MDP).

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (1₃) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition of the present invention can be administered by a variety of methods known in the art. As will be appreciated by the skilled artisan, the route and/or mode of administration will vary depending upon the desired results.

The term “administration,” is intended to include any route of introducing into a subject a macrophage-binding compound of the invention which allows the compound to perform its intended function (i.e., macrophage reduction and/or inhibition). Examples of routes of administration which can be used include injection (subcutaneous, intravenous, parenterally, intraperitoneally, intrathecal, etc.), oral, inhalation, rectal and transdermal. The pharmaceutical preparations are of course given by forms suitable for each administration route. For example, these preparations are administered in tablets or capsule form, by injection, inhalation, eye lotion, ointment, suppository, etc.; administration by injection, infusion or inhalation; topical by lotion or ointment; and rectal by suppositories. The injection can be bolus or can be continuous infusion. Depending on the route of administration, the macrophage-binding compound can be coated with or disposed in a selected material to protect it from natural conditions which may detrimentally effect its ability to perform its intended function. The macrophage-binding compound can be administered alone, or in conjunction with either another agent as described above or with a pharmaceutically acceptable carrier, or both. The macrophage-binding compound can be administered prior to the administration of the other agent, simultaneously with the agent, or after the administration of the agent. Furthermore, the compound can also be administered in a proform or inactive form (e.g., a macrophage-binding compound which includes a light-sensitive toxin) which is converted into its active metabolite, or more active metabolite in vivo, e.g., upon light exposure.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a macrophage-binding compound, such that it enters the subject's system and, thus, is subject to metabolism and other like processes, for example, subcutaneous administration.

In general, macrophage-binding compounds of the invention are administered locally to treat or diagnose disorders characterized by an abnormal number and/or function of macrophages within a particular area or region of the body (e.g., skin, lungs, joints, or muscle/nerve tissue). For dermatological applications, the compounds are preferably delivered or administered topically or by transdermal patches. Topical administration is preferred in treatment of skin lesions, including lesions of the scalp, lesions of the cornea (keratitis), and lesions of mucous membranes where such direct application is practical. Shampoo formulations are sometimes advantageous for treating scalp lesions such as seborrheic dermatitis and psoriasis of the scalp. Mouthwash and oral paste formulations can be advantageous for mucous membrane lesions, such as oral lesions and leukoplakia. A preferred way to practice the invention is to apply the macrophage-binding compound, in a cream or oil based carrier, directly to the lesion, e.g., the psoriatic lesion. Typically, the concentration of macrophage-binding compound in a cream or oil is 1-2%. In addition, intra-dermal administration is an alternative for dermal lesions such as those of psoriasis and wounds. Alternatively, an aerosol can be used topically. Oral administration is a preferred alternative for treatment of skin lesions and other lesions discussed above where direct topical application is not as practical, and it is a preferred route for other applications.

Additionally, the compositions can be delivered parenterally, especially for treatment of arthritis, such as psoriatic arthritis or rheumatoid arthritis, and for direct injection of skin lesions. Parenteral therapy is typically intra-dermal, intra-articular, intramuscular or intravenous. Intra-articular injection is a preferred alternative in the case of treating one or only a few (such as 2-6) joints. Additionally, the therapeutic compounds are injected directly into lesions (intra-lesion administration) in appropriate cases. As an alternative in the treatment of arthritis, the compounds of the invention can be administered systemically.

For the treatment of respiratory diseases, compositions of the invention can be administered by nasal aerosol or inhalation. Such compositions can be prepared as solutions in saline, employing benzyl alcohol or other suitable preservatives, absorption promoters to enhance bioavailability, fluorocarbons, and/or other conventional a solubilizing or dispersing agents.

In certain embodiments, compositions including the compounds can be administered systemically or locoregionally. For example, compositions of macrophage-binding compounds which include a light-sensitive moiety, e.g., a toxin or a linker, can be administered in such manner. Furthermore, some autoimmune conditions such as multiple sclerosis are preferentially treated by either of locoregional or systemic administration of the compositions of the invention.

Powders and sprays can contain, in addition to compounds of the invention, carriers such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Regardless of the route of administration selected, the macrophage-binding compound, which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., (1984) J. Neuroimmunol. 7:27). Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations of the present invention include those suitable for topical, dermal or epidermal administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent.

Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given alone or as a pharmaceutical composition containing, for example, 0.01 to 99.5% (more preferably, 0.1 to 90%) of active ingredient in combination with a pharmaceutically acceptable carrier.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a compositions of the invention will be that amount of the compound which is the lowest dose effective to produce a therapeutic effect. Such an effective dose will generally depend upon the factors described above. It is preferred that administration be local, e.g., topical, subcutaneous, intradermal, preferably administered proximal to the site of the target. If desired, the effective daily dose of a therapeutic compositions may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the compounds of the invention can be formulated to ensure proper distribution in vivo. In one embodiment, the macrophage-binding molecules can be encapsulated into liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). For example, certain embodiments, it is preferable to use single chain antibodies against an Fc receptor (scFv), for example, H22 scFv, to target the compounds of the invention to Fc-bearing macrophages. Protocols for preparing liposome encapsulated scFv fragments are described in de Kruif, J. et al. (1996) FEBS 399: 232-236. For example, lipid-modified H22 scFv can be coupled to liposomes composed of egg phosphatidylcholine (EPC), egg phosphatidylglycerol (EPG), cholesterol and, optionally, rhodamine-phospatidylethanolamine (rhodamine) as a fluorescent bilayer marker, at a molar ratio of 10:1:5:0.01, by diluting mixed micelles containing n-octyl β-D-glucoside, lipid and lipid modified scFv to a level far below the critical micelle concentration of the detergent. Incorporation of scFv molecules in the liposomes can be verified by SDS-PAGE.

A “therapeutically effective dosage” is that dosage which reduces the number of macrophages within a selected treatment area relative to an untreated control, or which inhibits activity of macrophages within a selected area so that, for example, they no longer proliferate or contribute to inflammatory responses within the area. As a consequence, the symptoms of the macrophage-mediated disease are improved. The ability of compounds of the invention to kill or inhibit a population of macrophages can be evaluated in an animal model system, such as a transgenic animal expressing a human Fc receptor as described in the Examples herein. Alternatively, these functions can be evaluated in in vitro assays known to the skilled practitioner. A therapeutically effective amount of a therapeutic compound can decrease the macrophage cell population or activity, or otherwise ameliorate symptoms in a subject. One of ordinary skill in the art would be able to determine such amounts based on such factors as the subject's size, the severity of the subject's symptoms, and the particular composition or route of administration selected.

The composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as manitol or sorbitol, and sodium chloride in the composition. Long-term absorption of the injectable compositions can be brought about by including in the composition an agent which delays absorption, for example, aluminum monostearate or gelatin.

Uses and Methods of the Invention

Macrophage-binding compounds of the present invention have several diagnostic, therapeutic and research utilities. They can be administered to cells in vitro (in culture), ex vivo, or in vivo (in a subject), to treat, diagnose or study a variety of disorders.

In one embodiment, a method of depleting (e.g., reducing the number) or inhibiting the activity of macrophages in a selected treatment or diagnostic area is provided. The method involves contacting the selected area with the macrophage-binding compound in an amount sufficient to achieve the aformentioned result. As used herein, the terms “selected area” or “local area” collectively refer to any selected sample of tissue or cells (either in vitro or in vivo) which contain, or may contain, macrophages which contribute to a disorder, such as a localized area of the human body (skin, lungs, joints, etc.) or a tissue culture sample. The contacting can occur in vitro (e.g., cells in culture) or in vivo (e.g., by administering the compounds of the invention to a subject).

As used herein, the term “subject” is intended to include human and non-human animals. Preferred human animals include a human patient having a disorder characterized by aberrant activity of a macrophage cell, e.g., a skin macrophage cell. The term “activity” is intended to include all biological functions of a macrophage cell, including proliferation, differentiation, survival, growth factor or cytokine secretion, among others. The term “non-human animals” of the invention includes all vertebrates, e.g., mammals and non-mammals, such as non-human primates, sheep, dog, cow, chickens, amphibians, reptiles, etc.

Macrophage-binding compounds of the invention can be initially tested in vitro. For example, the activity of these molecules killing and/or modulating, e.g., reducing, macrophage activity can be assayed in macrophage-derived cell lines, cultured differentiated blood monocytes, and primary culture systems. Protocols for assaying in vitro activity of macrophage-binding compounds can be found, for example, in Immunopharmacology of Macrophages and Other Antigen-presenting Cells (ISBN 0-12-137800-4, 1994, Academic Press Limited). For example, primary skin macrophage cultures can be established from skin cells derived from healthy and dermatologic subjects. Macrophage activity, e.g., cell proliferation or cytokine secretion, can be assayed at specific time intervals after the addition of a range of concentrations of the compounds of the present invention. In one embodiment, ‘punch biopsies’ obtained from healthy and dermatologic subjects can be used. Punch biopsies can be cultured either submerged, or with the epidermal side surfaced in culture medium, to which the compounds of the invention can be added. Following culture with the macrophage-binding compounds of the invention, the effect(s) of these compounds in macrophage activity can be assayed immunohistochemically or by ELISA, RIA or EIA.

Protocols for detecting changes in cell proliferation, e.g., thymidine or BrdU incorporation assays, are known in the art. Preferred macrophage-binding compounds of the invention decrease or eliminate macrophage activity. Protocols for detecting changes in cytokine concentration can be detected via a variety of immunoassays, such as enzyme-linked immunoassay (ELISA), enzyme immunoassay (EIA) or radioimmunoassay (RIA) which are known in the art (see e.g., Keler, T. et al. (1997) Cancer Research 57: 4008-14). Exemplary cytokines that can be assayed include: granulocyte/macrophage colony stimulating factor (GM-CSF), granulocyte colony-stimulating factor (G-CSF), macrophage colony-stimulating factor (M-CSF), interleukins 1-12 (IL-1 to IL-12), and TNF-α. The concentration of a cytokine can be measured using an EIA by detecting the interaction of the cytokine with an antibody, which is in turn conjugated to an enzyme. The activity of the enzyme is detected by the reaction with an appropriate substrate, preferably a chromogenic substrate, in such a manner as to produce a chemical moiety which can be detected, for example, by spectrophotometric, fluorimetric or by visual means (Voller, “The Enzyme Linked Immunosorbent Assay (ELISA),” Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). Enzymes which can be used to detectably label the antibody are described above. The detection can be accomplished by colorimetric methods which employ a chromogenic substrate for the enzyme. Detection may also be accomplished by visual comparison of the extent of enzymatic reaction of a substrate in comparison with similarly prepared standards.

Detection of a cytokine may also be accomplished using a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

It is also possible to label the anti-cytokine antibody with a fluorescent compound. When the fluorescently labeled antibody is exposed to light of the proper wave length, its presence can then be detected. Among the most commonly used fluorescent labeling compounds are fluorescein isothiocyanate, rhodamine, phycoerythrin, phycocyanin, allophycocyanin, o-phthaldehyde and fluorescamine. The antibody can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA). The antibody also can be detectably labeled by coupling it to a chemiluminescent compound. The presence of the chemiluminescent-tagged antibody is then determined by detecting luminescence that arises during the course of a chemical reaction. Examples of particularly useful chemiluminescent labeling compounds are luminol, isoluminol, theromatic acridinium ester, imidazole, acridinium salt and oxalate ester. Likewise, a bioluminescent compound may be used to label the antibody. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Macrophage-binding compounds also can be tested in vivo. For example, these compounds can be tested using mice expressing human Fc receptors as described in the Examples herein. In one embodiment, macrophage-binding compounds can be injected intradermally into these transgenic mice. Vehicle-injected controls can be processed in parallel. Chronic cutaneous inflammation can be induced experimentally in these mice by repeated topical application of 5% sodium lauryl sulfate. The effects of these compounds can be monitored immunohistochemically, e.g., macroscopically or clinically, at various time intervals after injection.

The macrophage-binding compounds of the invention can be used in the treatment of disorders characterized by aberrant macrophage activity or numbers. The term “aberrant” refers to a macrophage density within a selected site which is different (e.g., higher) than that found in the same area in normal, healthy patients. The term “aberrant” also includes abnormal macrophage activity, such as abnormally high cell proliferation or cytokine secretion. Accordingly, in one embodiment, the invention provides a method of treating or prophylactively preventing disorders characterized by aberrant numbers or activity of macrophages in a selected area, comprising administering to a subject, generally in the local area needing treatment, a pharmaceutical composition containing one or more macrophage-binding compounds.

Macrophage-binding compounds are generally used as targeting agents to deliver cytotoxins (e.g., drugs) to Fc receptor-bearing macrophages. In one embodiment of the invention, the cytotoxin is encapsulated within a liposome which itself is targeted to Fc receptor-bearing macrophages. Thus, the macrophage-binding compound comprises an anti-Fc receptor binding portion linked to a liposome containing a cytotoxin. In a preferred embodiment, the anti-Fc receptor binding portion is a single chain antibody directed against an Fc receptor (scFv), such as H22 scFv. The anti-FcR scFv is linked or inserted into the lipid bilayers of the liposome in a manner which allows the scFv still to recognize and bind to Fc receptors outside the liposome. This can be done using known protocols, such as those described by de Kruif, J. et al. (1996) FEBS 399: 232-236. The end result is an FcR targeted cytotoxin which is delivered to cells in the form of a liposome.

As used herein, a “therapeutically effective amount” of a macrophage-binding compound refers to an amount of a compound which is effective, upon single or multiple dose administration to the subject, at inhibiting the growth of the cells, or an improvement in the clinical symptoms in the absence of such treatment.

As used herein, “a prophylactically effective amount” of a compound refers to an amount of a macrophage-binding compound which is effective, upon single- or multiple-dose administration to the patient, in preventing or delaying the occurrence of the onset or recurrence of a macrophage-mediated disease state.

The terms “induce”, “inhibit”, “potentiate”, “elevate”, “increase”, “decrease” or the like, e.g., which denote quantitative differences between two states, refer to at least statistically significant differences between the two states. For example, “an amount effective to inhibit growth of the macrophage cells” means that the rate of growth of the cells will at least statistically significantly different from the untreated cells.

Macrophage-binding compounds of the invention can be used to treat a variety of macrophage-mediated diseases. These diseases are not necessarily characterized solely by aberrant macropage numbers and/or activity, but they each involve undesired macrophage activity which is harmful to patients. In one embodiment, the compounds are used to treat autoimmune diseases including, for example, diabetes mellitus, arthritis (including rheumatoid arthritis, juvenile rheumatoid arthritis, osteoarthritis, psoriatic arthritis), multiple sclerosis, encephalomyelitis, diabetes, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis). Downmodulation of immune activity will also be desirable in cases of allergy such as, atopic allergy.

Exemplary of preferred autoimmune/dermatological disorders for which the subject method may be used as part of a treatment regimen include: psoriasis, atopic dermatitis, multiple sclerosis, scleroderma and cutaneous lupus erythematosis. For example, the methods and compositions of the invention can be used to treat atopic dermatitis (AD). Without being bound by theory, it is believed that during the acute phase of cutaneous inflammation in AD, the phenotype of local T cells switches from an initial Th2 type to a Th1 type in the chronic phase. At this timepoint, an increase in IL-12 production in the lesion is found, together with a strong influx of activated inflammatory macrophage. Macrophages are potent producers of IL-12 which induces T cells to produce IFN-γ, which in turn is a potent macrophage activator (Thepen, T. et al. (1996) J Allergy Clin. Immunol. 97: 828-837; Grewe, M. et al. (1998) Immunol. Today 19:359361). Such positive feedback potentially creates a vicious circle, which by itself may be capable of maintaining local inflammation without the necessity of external stimuli. Other such mechanisms, resulting in a continual allergen non-specific response, resulting from dysregulation of macrophage are plausible, considering the regulatory potential of macrophages. The selective, localized elimination of inflammatory macrophages by targeting an Fc receptor, e.g., FcγRI, described in the Examples below makes the compositions of the invention useful for reducing or eliminating the positive feedback loop created upon macrophage secretion, and thus treating diseases such as AD.

Additional examples of diseases that can be treated via therapeutic methods of the invention include infectious diseases, e.g., HIV infections, respiratory conditions, e.g., Chronic Polymorphic Light Dermatosis (CPLD), Chronic Obstructive Pulmonary Diseases (COPD), for example, allergic asthma and Sarcoidosis, and inflammatory reactions such as those observed in open wounds or burn wounds.

In other embodiments, the compositions and methods of the present invention can be used in cosmetic applications. For example, the macrophage-binding compounds can be applied locally (e.g., topically) to the skin to delay and/or prevent the aging process of the skin.

The therapeutic methods of the present invention can be performed in conjunction with other techniques for removal of macrophage cells. For example, therapy using macrophage-binding compounds of the invention can be used in conjunction with surgery, chemotherapy or radio-therapy.

Macrophage-binding compounds of the invention can also be used to modulate FcγR levels on effector cells, such as by capping and elimination of receptors on the cell surface. Mixtures of anti-Fc receptors can also be used for this purpose.

The present invention further provides a kit comprising one or more dosages of a macrophage-binding compound and instructions for use.

In other embodiments, combinations of macrophage-binding compounds of the invention can be used to selectively kill or reduce the activity of macrophages, e.g., a combination of a first compound having at least one antigen binding region specific for an FcR and a toxin, and a second compound having an antigen binding region to a different epitope of the FcR receptor or a different Fc receptor, e.g., an Fcα receptor. In certain embodiments, a second macrophage-binding compounds of the invention can be used in conjunction with the first. For example, this second macrophage-binding compound can have at least one antigen binding region specific for an IgA receptor, e.g., Fcα receptor, IgE receptor, e.g., Fcε receptor, an Fcδ receptor and/or an Fcμ receptor.

Prior to administering macrophage-binding compounds to a subject, the subject can be pre-treated with an agent that modulates, e.g., enhances or inhibits, the expression or activity of Fcγ receptors, by for example, treating the subject with a cytokine. Preferred cytokines for administration during treatment with the macrophage-binding compound include of granulocyte colony-stimulating factor (G-CSF), granulocyte-macrophage colony-stimulating factor (GM-CSF), interferon-γ (IFN-γ), and tumor necrosis factor (TNF).

Macrophage-binding compounds of the invention can also be used diagnostically in vitro and in vivo to detect and/or measure macrophage populations by measuring levels of Fc receptor binding. For example, as shown in the Examples provided herein, abundant expression of FcγRI is detected in the dermis of both acute and chronic cutaneous inflammation in humans. Therefore, the macrophage-binding compounds described herein can be used to diagnose such inflammatory conditions. For such uses, the compound can be linked to a molecule that can be detected. The detectable label can be, for example, a radioisotope, a fluorescent compound, an enzyme, or an enzyme co-factor. Accordingly, in another embodiment, the invention provides a method of diagnosing in vitro or in vivo disorders characterized by aberrant numbers of macrophages (e.g., macrophage proliferation) and/or Fc receptor expression (e.g., increased number of cells expressing an Fc receptor and/or increased Fc receptor expression in a given cell). By measuring the level of binding of the compounds of the invention in a given test sample or within a localized area, the presence of macrophages within the area or sample can be deduced, provided that the anti-Fc receptor component of the compound is specific for macrophage Fc receptors. This can be done by (i) obtaining a body sample, such as a body fluid, tissue (e.g., a skin sample) or biopsy from a patient; (ii) contacting the body sample with a macrophage-binding compound of the invention or a fragment thereof; (iii) determining the level of binding of said macrophage-binding compound to the body sample; (iv) comparing the amount of molecule bound to the body sample to a control sample, e.g., a biological sample from a healthy subject, or to a predetermined base level, so that a binding greater than the control level is indicative of the presence of a macrophage disease, e.g., skin disease. Preferably, the level of Fc receptor expression is detected primarily on the macrophage cell population relative to other Fc receptor-expressing cells. Protocols for in vivo and in vitro diagnostic assays are provided in PCT/US88/01941, EP 0 365 997 and U.S. Pat. No. 4,954,617.

The following invention is further illustrated by the following examples, which should not be construed as further limiting. The contents of all references, pending patent applications and published patents, cited throughout this application are hereby expressly incorporated by reference.

### EXAMPLES

Materials and Methods

The following methodologies were used in the studies described below. The terms macrophage-binding compounds, CD64 immunotoxins (CD64 IT), or immunotoxins (IT) are used interchangeably herein.

Monoclonal Antibodies

The examples below describe the use of an anti-CD64 (anti-FcR) antibody corresponding to a humanized form of monoclonal antibody 22 (H22), described in U.S. Pat. No. 5,635,600, which is incorporated by reference. The production and characterization of the H22 antibody is described in Graziano, R. F. et al. (1995) J. Immunol 155 (10): 4996-5002 and PCT/US93/10384. The H22 antibody producing cell line was deposited at the American Type Culture Collection on Nov. 4, 1992 under the designation HA022CL1 and has the ATCC accession number CRL 11,177.

Other specific anti-CD64 antibodies which can be used in the methods and compositions of the invention are murine antibodies mAb 32.2, mAb 44, mAb 62 and mAb 197. The hybridoma producing mAb 32.2 is available from the American Type Culture Collection. ATCC accession number HB9469. The preparation of mAb 197-Ricin A conjugates is described in the Examples below.

The anti-FcR mAbs were purified from each respective hybridoma supernatant by protein A affinity chromatography (Bio-Rad, Richmond, Calif.).

Immunohistochemical Staining

A: CD64 Staining

Biopsies were cut into 6 μm sections on a freezing microtome and mounted on coated slides. After drying overnight, the sections were fixed for 10 minutes with dry acetone and air dried. Slides were incubated with FITC conjugated 10.1 (Serotec 1:40) in PBS 2% normal mouse serum (NMS) for 45 min. Slides were washed three times for 5 minutes with PBS, 0.05% Tween, after which alkaline phosphatase (AP) conjugated sheep anti FITC (Boehringer Mannheim, 1:400) in PBS (1% Human AB serum, 1% NMS for 30 min). After washing twice in PBS/Tween and once in Tris-HCl (0.1M, pH 8.5), AP activity was demonstrated using naphtol AS-BI phosphate (sodium salt, 50 mg/100 ml; Sigma) as substrate and new fuchsin (10 mg/100 ml; Merck, Whitehouse Station, N.J.) as chromogen dissolved in 0.1M Tris HCl, pH 8.5, resulting in pink/red staining. Endogenous AP activity was inhibited by addition of levamisole (35 mg/100 ml, Sigma) to the reaction mixture. Slides were lightly counterstained with hematoxylin.

B: Markers

Sections were fixed in dry acetone with H₂O₂ (30%, 100 μl/100 ml) for 7 min. Slides were incubated with primary rat antibodies in optimal dilution for 45 min in PBS 2% NMS. The following antibodies were used to stain macrophages: MOMA-2 (Kraal, G. et al. (1987) Scand. J. Immunol. 26: 653-661); dendritic cells: NLDC145 (Kraal, G. et al. (1986) J. Exp. Med. 163: 981-997); T cells: KT3 (Tomonari, K. (1988) Immunogenetics 28:455-458). After washing three times (5 minutes in PBS, 0.05% Tween 20), incubation with peroxidase labeled rabbit anti rat conjugate (DAKO, 1:200), in PBS (1% Human AB serum, 1% NMS) followed for 30 minutes. After rinsing twice with PBS and once with NaAc (0.1M, pH 5.0), PO activity was revealed using H₂O₂ as substrate and DAB (Sigma) as chromogen, resulting in brown staining.

Animal Studies

Induction of cutaneous inflammation, Immunotoxin injections, and Biopsies. In the experiments described herein, transgenic FVB/N mice expressing human FcγRI were used (Heijnen, I. A., et al. (1996) J. Clin. Invest. 97:331 338). Nontransgenic littermates served as controls. To induce chronic cutaneous inflammation, an area of 1.5 by 1.0 cm on both flanks of the mice was shaved and the irritant Sodium Lauryl Sulfate (SLS) (5% in saline) was applied epicutaneous daily for ten consecutive days.

Animals were anaesthetized with 20 μl of a 4:3 mixture of Aescoket (Aesculaap, Gent, Belgium) and Rompun (Bayer, Leverkussen, Germany), intramuscularly injected. Two adjacent intradermal injections, (10 μl each, 2×10⁻⁸ M, referring to the Ricin-A moiety, in saline) were administered. For control purposes, identical saline injections were administered contralaterally.

Animals were anaesthetized as described above and 3 mm punch biopsies were taken, snap frozen in liquid nitrogen and stored at −70° C. prior to use. The skin was closed with one suture.

Punch biopsies (3 mm) were taken under local anesthesia (1% lidocaine) from lesional AD skin (n=3), 24 h APT (n=3), 48 h SLS (n=2), and 72 h WB challenged PLE skin. Biopsies were snap frozen in liquid nitrogen and stored at −70° C. prior to use.

### Example I

### Preparation of CD64 Immunotoxins

The CD64 monoclonal antibodies 197 (Guyre, P. M., et al. 1989. J. Immunol. 143: 1650-1655) and H22 (Graziano, R. F., et al. 1995. J. Immunol. 155:4996-5002) were conjugated to de-glycosylated Ricin A (30 KDa, Sigma) using using a suitable linker (such as the heterobifunctional cleavable crosslinker N-succinimidyl 3-(2-pirydyldithio) proprionate (SPDP) (Pierce) under GLP conditions according to the manufacturers' instruction. Briefly, SPDP was conjugated to the CD64 mAb, e.g., H22, then the molar ratio of mAb-PDP was determined. After determining the molar ratio of mAb-PDP, Ricin A was added. Free PDP groups and free Ricin A chains were inactivated and the mixture was purified by size exclusion chromatography. The purity of H22-Ricin A conjugates was further checked by SDS-PAGE. H22-Ricin A conjugates were sterilized using an 0.2 μm filter. All preparation steps were performed under Good Manufacturing Practice conditions.

### Example II

### Effective Cell Killing of Macrophages Using CD64 Immunotoxins

Constitutive expression of FcγRI is primarily restricted to cells of the myeloid lineage, and is strongly upregulated under proinflammatory and inflammatory conditions (Velde, A. A., et al. (1992) J. Immunol. 149:4048-4052; Schiff, D. E., et al. (1997) Blood 90:3187-3194). The ability of FcγRI to rapidly and efficiently mediate endocytosis makes this receptor an effective target for activated inflammatory macrophages (Heijnen, I. A et al. (1996) J. Clin. Invest. 97:331 338). Several immunotoxins against hFcγRI were prepared as described in Example I using conjugates of the toxin Ricin-A and CD64 antibodies.

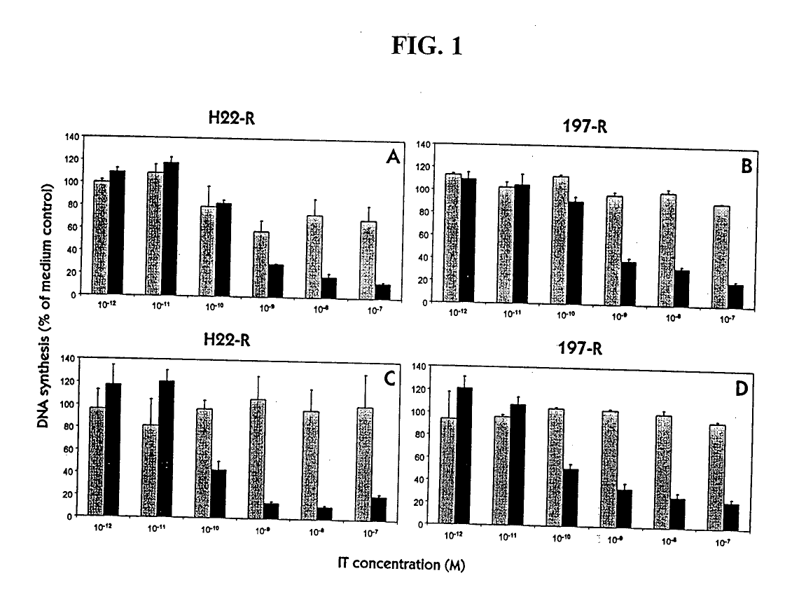

To establish the efficiency of these conjugates in inducing macrophage killing, the cultured human promonocytic cell line U937, either unstimulated, or stimulated with IFN-γ, was examined in the presence or absence of the compositions of the present invention (FIGS. 1, A and B). Culture conditions and stimulation of U937 cells with cytokines is described in Guyre, P. M., et al. (1983) J. Clin. Invest. 72:393-397. Briefly, U937 cells were cultured in the presence of 300 U/ml IFNγ for 24 hours to upregulate Fc γRI expression. FcγRI levels were monitored by flow cytometry. In addition, IIA1.6 cells, either non-transfected or transfected with FcγRIa cDNA were tested. IIA1.6 cells are derived from the murine A20 B cell lymphoma and were recently shown to belong to a distinct subset of CD5+B cell/macrophage cells (van Vugt, M. J., et al. 1998. Clin. Exp. Immunol. 113:415-422).

The cytotoxic efficacy of the CD64 immunotoxin (IT) was assessed by measuring the inhibition of [³H]Thymidine incorporation in a concentration-dependent fashion (Post, J et al. Leuk. Res. 19:241-247). Briefly, cells were seeded at 5×10⁴ cells/well in a 96 wells round bottom plate and incubated with CD64 IT for 72 hours in concentrations ranging from 10⁻¹² to 10⁻⁷ M referring to the ricin moiety. Cells were pulsed for 4 h with [³H]-Thymidine (1 μCi) and subsequently harvested on glasswool filters and counted on a beta plate scanner. All incubations were performed in culture medium supplemented with 2% human AB serum to block the Fc-binding site of FcγRI, thereby allowing binding of the IT by its antigen recognition site only. Cell numbers seeded were chosen such, that [³H]-Thymidine incorporation was a linear function of the number of cells. Background values of [³HlThymidine incorporation were obtained by incubation with 0.1 mM cycloheximide.

Results were expressed as percentage [³H]Thymidine incorporation compared to mock-treated cells. In FIGS. 1A-1B, the bar graphs represent the percentage of [³H]-Thymidine incorporation as compared with that of medium control (±SEM). The dose dependent decrease in [³H]-Thymidine incorporation as a function of increasing concentrations of H22-R or 197-R shows the cytotoxicity of the immunotoxins on the stimulated U937 cells. For panels 1C-1D, the bar graphs represent the percentage of [³H]-Thymidine incorporation as compared with that of medium control (±SEM). The dose dependent decrease in [³H]-Thymidine incorporation with resepect to increasing concentrations of H22-R or 197-R shows the cytotoxicity of the immunotoxins on hFcγRI-transfected IIA1.6 cells. This demonstrates the specificity of both IT for hFcγRI-expressing cells.

The two immunotoxins tested were potent inducers of cell killing. However, H22 Ricin-A (H22-R) was on the whole more effective than 197 Ricin-A (197-R) in inducing cell killing, especially on unstimulated cells. Incubation with Ricin-A alone at 10⁻⁸ and 10⁻⁹ M had no significant effect (88.9±14.2 and 100.4±13.5 percent, respectively). Furthermore, no significant effect of either IT was found on the non-transfected IIA1.6 cells, in contrast to the effective killing of hFcγRI-transfected IIA1.6 cells detected using either of these ITs (FIG. 1, panels C and D).

These results demonstrate both the efficacy and specificity of CD64 IT in killing hFcγRI-expressing cells in vitro. On the basis of these experiments, H22-R was used at a concentration of 2×10⁻⁸ M in the in vivo experiments described below.

### Example III

### Induction of Apoptosis by CD64-Immunotoxins

To establish whether the cytotoxic effect of H22 Ricin-A was due to apoptosis induction, propidium iodide staining in hypotonic buffer was performed. In this assay segmented apoptotic nuclei are recognized by subdiploid DNA content. To conduct these experiments, nuclear fragmentation was detected using propidium iodide staining as described in Nicoletti, I., et al. (1991) J. Immunol. Methods 139:271-279. In short, cells were incubated with IT and harvested at different timepoints. Cells were fixed with ethanol at −20° C., incubated with extraction buffer (0.05M Na₂HPO₄; 0.0025M citric acid; 0.1% Triton X-100; 20 μg/ml propidium iodide). Propidium iodide fluorescence was analyzed using a Fluorescent Activated Cell Sorter (FACScan) flow cytometer (Beckton and Dickinson, San Jose, Calif.).

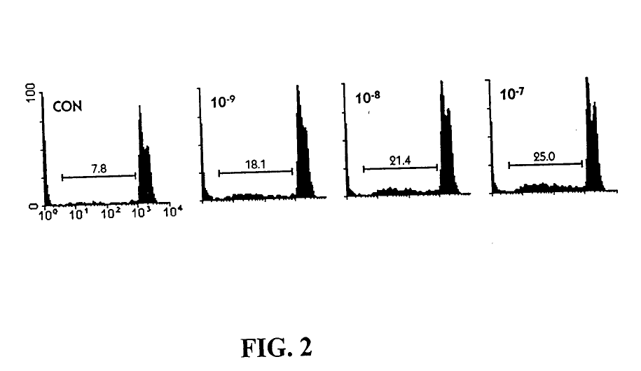

As shown in FIG. 2, apoptotic nuclei were detected in IT-treated cultures relative to control. In this experiment, U937 cells were stimulated with IFNγ and incubated for 6 h with different concentrations of H22-R. Apoptotic nuclei were detected as early as 2 hours after IT exposure, and was still evident after 16 hours of treatment. This finding shows that the cytotoxic effect of H22 Ricin-A IT results from the induction of apoptosis. Apoptosis-mediated cell killing limits the potential damaging effects by depletion of hFcγRI-expressing cells in vivo. In addition, the long lasting cell killing induced by H22-R (even after 16 hours) suggests the practicability of H22-R as IT to deplete hFcγRI-expressing cells in vivo.

### Example IV

### Detection of FcγRI-Expressing Cells in Chronic Cutaneous Inflammation in Humans

The staining ability of another CD64 monoclonal antibody, 10.1(Dougherty, G. J et al. 1987. Eur. J. Immunol. 17:1453-1459), was tested after pre-incubation of sections with H22 antibodies and in the presence of varying concentrations of H22 antibody. Since the 10.1 and H22 recognize different epitopes on hFcγRI, no significant change in staining intensity, or pattern was detected upon simultaneous incubation. Based on these results, the 10.1 antibody was used in all experiments involving immuno-histochemical evaluation of collected tissues.

To examine the presence of FcγRI-expressing cells in chronic cutaneous inflammation in humans, biopsies from chronically inflamed skin from patients with atopic dermatitis (AD) were collected. The diagnosis of AD was made according to the criteria of Hanifin and Raijka (Hanifin, J. M., and Rajka, G. (1980) Acta Derm. Venereol. (Stockholm) 92:44-47). Atopy Patch Test (APT) was performed as described in Langeveld-Wildschut, E. G., et al. (1995) J. Allergy Clin. Immunol. 96:66-73. In short, skin was tape stripped ten times and the allergen Dermatophagoides pteronyssinus (Haarlem's Allergenen Laboratorium, Haarlem, The Netherlands; 80, μL, 10,000 AU/ml) was applied using Leucotests (Beiersdorf, Hamburg, Germany) on clinically normal skin of the back of patients diagnosed with AD. On analogous skin, Sodium Lauryl Sulfate (SLS, Sigma, 0.1% in saline) was applied in a similar fashion. Polymorphic Light Eruption (PLE) was diagnosed on the basis of a polymorphic clinical picture, with presence of papules and vesicles, severe itching and clinical response after WA and/or WB irradiation. Previously unexposed skin was irradiated with 6 minimal erythema dose, using a Philips TL12 UVB source.

Sections from human skin were immunohistochemically stained using FcγRI antibodies. FcγRI-expressing cells were detected resulting as pink/red staining and counterstained with hematoxiline. In normal unaffected skin, few cells expressed FcγRI. These cells were located primarily in dermis. In contrast, abundant expression of FcγRI in dermis was observed in chronically lesioned skin, for example, atopic dermatitis skin. The stained cells were localized both in infiltrates and scattered through dermis. No significant staining in epidermis was observed. Next to these, biopsies from acute phase models, such as 24 hr after atopic patch test (APT), 72 hr after polymorphic light eruption skin (PLE), and 48 hr after treatment with Sodium Lauryl Sulfate (SLS), were collected. These biopsies gave similar results, however, the number of FcγRI-expressing cells was somewhat higher than in the chronically affected tissues. The very presence of large numbers of FcγRI-expressing cells in both acute and chronic phase is indicitive of a role for these cells in the inflammatory cutaneous response.

### Example V

### Establishment of a Murine Model for Chronic Cutaneous Inflammation

To determine whether elimination of inflammatory macrophages from skin is feasible and has a beneficial effect on cutaneous inflammation, the H22-R was tested in experimental animals. Induction of chronic cutaneous inflammation was studied using shaved skin of hFcγRI-transgenic mice and their nontransgenic littermates after repeated topical application of SLS. The expression pattern, gene regulation and function of hFcγRI in these mice mirrors that in humans (Heijnen, I. A., et al. (1996) J. Clin. Invest. 97:331 338). Several protocols were tested and daily application of 5% SLS for ten days proved adequate as described in the section entitled Materials and Methods.

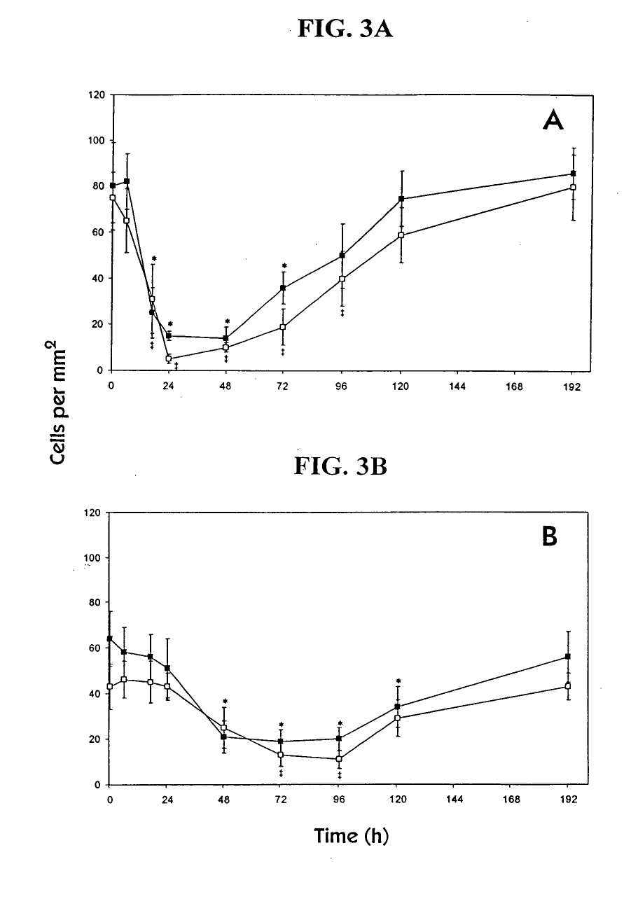

Low numbers of T cells, dendritic cells, and macrophages were detected in normal, untreated skin (5±4; 7±4 and 15±3 per mm² respectively). In addition, few hFcγRI-expressing cells were detected in normal, untreated skin (5±2 per mm²), and the distribution resembled that of normal unaffected human skin. Treatment with SLS resulted in thickening of epidermis and a vast dermal infiltrate consisting of T cells, dendritic cells, and macrophages (FIG. 3A). For these experiments, a single intradermal injection of IT was administered into chronically inflamed skin and at different intervals punch biopsies were taken and stained immunohistochemically. The number of cells expressing hFcγRI also increased dramatically (FIG. 3A) (75±11 per mm²) and like in chronically affected human skin, these cells were primarily distributed in the dermis. There was no significant difference in cellular composition between the hFcγRI-transgenic and non-transgenic mice. In the latter however, no significant cells staining for hFcγRI were observed. No detectable presence of either hFcγRI-expressing cells or macrophages was observed after injection with H22-R only.

The similarities with respect to cellular composition and hFcγRI expression between chronically inflamed human skin and the SLS induced inflammation in hFcγRI-transgenic mice make this a suitable model to study the role of hFcγRI expressing cells during chronic cutaneous inflammation. This model in combination with the H22-R IT was used in the Examples set forth below.

### Example VI

### Effective Depletion of FcγRI-Expressing Macrophages In Vivo

To determine whether H22-R was as effective in killing hFcγRI-expressing cells in vivo as it proved to be in vitro, H22-R was injected intradermally in mice treated with SLS. Chronic cutaneous inflammation was induced in the human FcγRI-expressing transgenic mice by repeated topical application of an irritant, 5% sodium lauryl sulfate as described in the section entitled Materials and Methods, supra. Two adjacent 10 μl intradermal injections of 2×10⁻⁸ M (3 μg of H22 and 0.6 μg of Ricin A) were administered once to SLS treated skin of hFcγRI-transgenic and nontransgenic mice. Identical vehicle control injections were administered contralaterally. SLS application was continued while at different timepoints skin samples, draining lymph nodes, liver, and spleen were collected for immuno-histochemical analysis.

The localized nature of the intradermal injections was examined by detecting uptake of carbon particle by macrophages. A cross-section of murine skin after intradermal injection of carbon particles revealedthe presence of carbon particles primarily in the dermis, but not below cutaneous musculature. This distribution demonstrates the localized nature of the intradermal injections.

A representative immunohistochemical cross-section of skin of human FcγRI-expressing transgenic mouse after repeated topical applications of sodium lauryl sulfate, and intradermal injection with vehicle control or Ricin A-H22 revealed the thickening of the epidermis and large number of infiltrating cells in the dermis 24 hours after treatment. This pattern of staining indicates chronic inflammation induced by the irritant. The majority of the infiltrating cells detected were FcγRI-positive macrophages (stained in pink). In contrast, the staining of Fcγ receptor-expressing infiltrated cells was significantly reduced 24 hours after injection of the immunotoxin Ricin A-H22.

The disappearance of hFcγRI-expressing cells from the skin was detected within 24 hours of exposure to IT (FIG. 3A). Despite continued SLS application, the depletion was complete till approximately 96 h after which repopulation occurred. Repopulation was complete only at 120 h (FIG. 3A). In draining lymph nodes, liver, and spleen, no significant changes in hFcγRI expression were observed. This observation emphasizes the fact that the effect remains restricted to the site of injection. In the vehicle control injected site and in the non-transgenic mice no significant changes were observed. The rapid and nearly complete disappearance of hFcγRI-expressing cells and their protracted absence from skin showed the practicability of the H22-R IT to eliminate hFcγRI-expressing cells in chronic cutaneous inflammation in vivo.

### Example VII

### Effect of Depletion of hFcγRI-Expressing Cells on Local Cutaneous Inflammation

Simultaneously with the reduction in hFcγRI-expressing cells, the abundance of MOMA-2-expressing macrophages was also diminished. This finding shows that injection of H22-R results in efficient depletion of inflammatory macrophages from affected skin (FIG. 3A). In contrast, no significant change in macrophage populations occurred in non-transgenic mice. This selective depletion confirms the specificity of H22-R in targeting and eliminating macrophages from skin.

To further assess the localized nature of the macrophage depletion, hematopoietic tissues such as lymph nodes, spleen, and liver were examined. No significant cell depletion by the immunotoxin was observed in other hematopoietic tissues. Identical treatment of non-transgenic littermates resulted in undetectable changes in any of the cell populations examined. These results indicate that the macrophage depletion was specific for human Fcγ RI-bearing cells and remained limited to the site of injection.

The specificity of the procedure in eliminating macrophages locally is further demonstrated by the disappearance, as early as within 24 hours, of macrophages, while no significant depletion was observed in dendritic cells, T cell populations, or Langerhans' cells during the timepoints examined. The H22-R injections had no direct effect on the numbers of T cells and dendritic cells (FIG. 3B). However, after the disappearance of hFcγRI-expressing macrophages, T cell and dendritic cell numbers started to decrease in the skin. The reduction of T cell and dendritic cell numbers is indicative of resolving local inflammation and thus a beneficial effect of deletion of inflammatory macrophages on local inflammation, even in the continued presence of the inflammatory stimulus (FIG. 3B).

These findings demonstrate the efficiency and specificity of the CD64 IT in depleting inflammatory macrophages from skin at the histological level. The subsequent disappearance of other inflammatory cells points to a deleterious role of macrophages in chronic cutaneous inflammation.

### Example VIII

### Local Macrophage Depletion Results in Clinical Improvement of the Skin

To determiner whether local macrophage depletion resulted in clinical improvement of the skin two parameters were measured: local skin temperature and erythema. Erythema is primarily due to increased capillary dilatation, and directly related to this increased skin temperature.

To detect changes in skin temperature induced by SLS application and IT injection, animals were immobilized by mild ether sedation and local temperature was measured using a skinprobe (Ellab A-H1, Denmark). To elucidate capillary dilatation and vascular leakage as parameter for inflammation, animals were sedated with ether and intravenously injected with a 1% Evans blue solution. After 15 minutes animals were sacrificed and skin was removed for assessment.

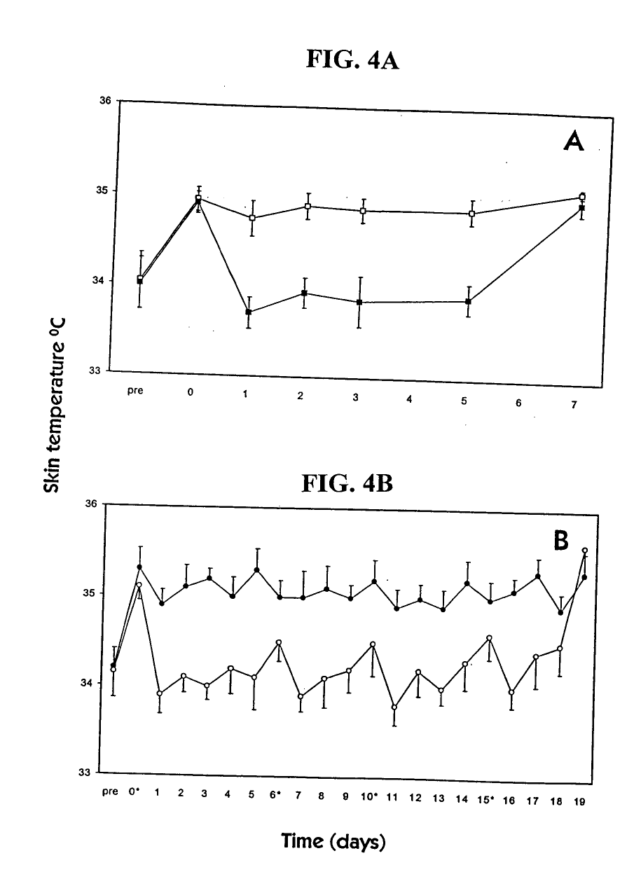

Using a small skin probe, local changes in skin temperature were measured in IT-treated and control animals. A rise in temperature after SLS treatment was detected confirming the induction of local inflammation. FIG. 4A is a bar graph depicting the effect of intradermal injection of H22-R on local skin temperature as a function of time. A drop in temperature reaching levels comparable to untreated, unaffected skin. This decrease in temperature in IT-treated animals was detected typically lasting 96 hours. After that time, the temperature increased again, reaching levels comparable to that prior to IT injection. These changes in temperature are indicative of the resolution of inflammation. Neither the vehicle control nor the nontransgenic mice showed a similar decrease in temperature. Moreover, a close temporal correlation between the disappearance of the macrophages and the decrease in local skin temperature was observed. Conversely, upon reappearance of the macrophages, an increase in temperature was detected. This findings are highly suggestive of a critical role of macrophages in local inflammation.

In mice, redness of the skin is difficult to assess due to the thinness of murine skin. To facilitate visualization of local capillary dilatation, Evans blue was intravenously injected into these animals. For these experiments, chronic cutaneous inflammation was induced in hFcγRI-transgenic mice (n=9) or non-transgenic mice (n=9) by epicutaneous application of SLS and IT or vehicle control was administered intradermally. Evans blue was injected intravenously at 24 h and 30 min later animals were killed and skin from the middle section was removed. Using this technique, the presence of an inflammatory response after SLS treatment was detected. No significant effect of the IT in capillary dilation was detected in non-transgenic mice or the vehicle control. At the H22-R-injected side of the latter however, the injection site itself was devoid of blue staining showing resolution of local inflammation. Moreover, the overall intensity of the blue staining was less at the H22-R-injected side.

### Example IX

### Prolonged Suppression of Inflammation In Vivo Upon Repeated Injections with CD64-IT

To establish whether the IT could be employed for a prolonged time, skin temperature was measured daily and upon increase the animals were again injected at the same site. During the experiment, SLS application was continued. FIG. 4B shows that inflammation could be controlled for at least 18 days in hFcγRI-transgenic mice injected with H22-R only. Vehicle control and non-transgenic mice did not show a significant decrease in temperature at any of the timepoints tested. Repeated injections with the IT demonstrated that it was possible to suppress inflammation for a prolonged period. This finding demonstrates the applicability of prolonged IT treatment in chronic cutaneous inflammation in patients. Taken together, these experiments show a beneficial effect of local macrophages elimination on the chronic cutaneous inflammation induced by SLS application.

In sum, the experiments described in the Examples herein show that activated macrophages can be eliminated selectively and efficiently eliminated using the methods and composition of the present invention, without significantly affecting other cutaneous or hematopoietic cell populations. Moreover, the effects of the immunotoxin remains primarily localized to the area of delivery, thus reducing negative systemic effect on other FcγR-expressing cells. The reduction in inflammation upon macrophage elimination underscores the importance of inflammatory macrophages as an agent in inducing and maintaining cutaneous inflammation. A reduction in the inflammation is detected at a histological level, as well as by decreases in clinical parameters such as local skin temperature and redness of the skin. Moreover, repeated application resulted in suppression of inflammation for prolonged periods. Prolonged effectivenes suggests the potential use of the methods and composition of the present invention in managing local cutaneous inflammation in patients suffering from chronic cutaneous diseases. This approach described herein may have wider applications since inflammatory macrophages are likely to play a key role in chronicity of other types of chronic inflammation, such as rheumatoid arthritis.

Staining for CD64 in human skin showed numerous FcγRI-expressing cells during both acute and chronic cutaneous inflammation. This observation indicates that targeting macrophages through FcγRI can indeed provide a new therapeutic approach for cutaneous inflammatory disease in humans. In fact, the effective reduction in SLS induced chronic inflammation in hFcγRI-transgenic mice showed herein supports potential therapeutic uses of these immunotoxins.

Equivalents

Those skilled in the art will recognize, or be able to ascertain using no more than routine experimentation, many equivalents of the specific embodiments of the invention described herein. Such equivalents are intended to be encompassed by the following claims.

We claim:

1. A method of selectively reducing the number or activity of macrophages within a localized area of tissue, comprising contacting the area of tissue with a macrophage-binding compound comprising (a) a first agent which binds to an Fc receptor at a site which is distinct from that bound by endogenous immunoglobulins; and (b) a second agent which kills or reduces the activity of the macrophages, wherein the first and second agents are different, and wherein the macrophage-binding compound is administered topically, intradermally or subcutaneously in a pharmaceutically acceptable carrier.

2. A method of treating a disease in a subject characterized by aberrant activity or numbers of macrophages within a selected area of the subject, comprising locally administering to the area a macrophage-binding compound comprising (a) a first agent which binds to an Fc receptor; and (b) a second agent which kills or reduces the activity of the macrophages, wherein the first and second agents are different, and wherein the macrophage-binding compound is administered topically, intradermally or subcutaneously in a pharmaceutically acceptable carrier.

3. The method of claim 2, wherein the agent which binds to an Fc receptor binds at a site which is not bound by an endogenous immunoglobulin.

4. The method of either of claims 1 or 2, wherein the Fc receptor is an Fcγ receptor (FcγR) or an Fcα receptor (FcαR).

5. The method of claim 4, wherein the Fcγ receptor is selected from the group consisting of FcγRI, FcγRII and FcγRIII.

6. The method of claim 5, wherein the Fcγ receptor is a human FcγRI.

7. The method of claim 4, wherein the Fc receptor is a human FcαR.

8. The method of either of claims 1 or 2, wherein the macrophage-binding compound comprises an anti-Fc receptor antibody conjugated to a toxin.

9. The method of claim 8, wherein the anti-Fc receptor antibody is an anti-Fcγ receptor antibody or a fragment thereof.

10. The method of claim 9, wherein the anti-Fcγ receptor antibody is a monoclonal antibody selected from the group consisting of mab 22, 32 and 197, or a fragment thereof.

11. The method of claim 9, wherein the anti-Fcγ receptor antibody is a humanized antibody H22 produced by the cell line having ATCC accession number CRL 1117 or a fragment thereof.

12. The method of claim 8, wherein the toxin is selected from the group consisting of Gelonin, Saporin, Exotoxin A, Onconase and Ricin A.

13. The method of claim 1, wherein the agent which kills or reduces the activity of the macrophages is encapsulated within a liposome.

14. The method of claim 13, wherein the agent which kills or reduces the activity of a macrophage is dichoromethylene diphosphonate (CL2MDP) or a derivative thereof.

15. The method of claim 13, wherein the agent which binds to an Fc receptor is a single chain antibody.

16. The method of claim 13, wherein the agent which binds to an Fc receptor is an anti-Fcγ receptor antibody or a fragment thereof.

17. The method of claim 13, wherein the agent which binds to an Fc receptor is a single chain anti-Fcγ receptor antibody or a fragment thereof.

18. The method of claim 1, wherein the contacting step occurs in culture.

19. The method of claim 2, wherein the disease is characterized by enhanced proliferation and/or growth factor secretion of the macrophage.

20. The method of claim 2, wherein the disease is selected from the group consisting of psoriasis, atopic dermatitis, scleroderma, cutaneous lupus erythematosis, Human Immunodeficiency Virus infection, multiple sclerosis, rheumatoid arthritis, Chronic Polymorphic Light Dermatosis, Chronic Obstructive Pulmonary Diseases, and Wegener's Granulomatosis.
